(19) United States
(12) Patent Application Publication
(10) Pub. No.: US 2003/0078373 A1
(43) Pub. Date: Apr. 24, 2003

(54) CHAPERONE FRAGMENTS

(76) Inventors: ALAN ROY FERSHT, CAMBRIDGE (GB); RALPH ZAHN, ZURICH (CH); MYRIAM MARLENNE ALTAMIRANO, CAMBRIDGE (GB)

Correspondence Address: FROMMER LAWRENCE & HAUG, 745 FIFTH AVENUE- 10TH FL., NEW YORK, NY, 10151, US

(21) Appl. No.: 09/276,455

(22) Filed: Mar. 25, 1999

(30) Foreign Application Priority Data
Sep. 26, 1997 (GB) 97/02652
Dec. 3, 1996 (GB) 96/02980
Sep. 26, 1996 (GB) 9620243.7

(51) Int. Cl. C07K 1/00; C07K 14/00; C07K 17/00

(52) U.S. Cl. 530/350

(57) ABSTRACT

The present invention provides a chaperone polypeptide having an amino acid sequence selected from at least amino acid residues 230-271 but no more than residues 150-455 or 151-456 of a GroEL, sequence substantially as shown in FIG. 7, or a corresponding sequence of a substantially homologous chaperone polypeptide, or a modified, mutated or variant sequence thereof having chaperone activity.

The present invention relates to chaperone polypeptides which are active in the folding and maintenance of structural integrity of other proteins. The invention also relates to nucleic acids encoding chaperone polypeptides vectors comprising these nucleic acids host cells modified with the nucleic acids or vectors so as to express the chaperone polypeptides. The invention further relates to methods of making chaperone polypeptides whether by synthetic or recombinant means pharmaceutical compositions comprising the chaperone polypeptides or nucleic acids encoding the same and the use of chaperone polypeptides in the treatment of disease or in the reconditioning of biologically active materials. The invention also relates to antibodies reactive against chaperone polypeptides and their use in medicine and diagnostics.

Chaperones are in general known to be large multisubunit protein assemblies essential in mediating polypeptide chain folding in a variety of cellular compartments. Families of chaperones have been identified, for example the chaperonin hsp60 family otherwise known as the cpn60 class of proteins are expressed constitutively and there are examples to be found in the bacterial cytoplasm (GroEL), in endosymbiotically derived mitochondria (hsp60) and in chloroplasts (Rubisco binding protein). Another chaperone family is designated TF55/TCP1 and found in the thermophilic archaea and the evolutionarily connected eukaryotic cytosol. A comparison of amino acid sequence data has shown that there is at least 50% sequence identity between chaperones found in prokaryotes, mitochondria and chloroplasts (Ellis R J and Van der Vies S M (1991) Ann Rev Biochem 60: 321-347).

A typical chaperonin is GroEL which is a member of the hsp60 family of heat shock proteins. GroEL is a tetradecamer wherein each monomeric subunit (cpn60m) has a molecular weight of approximately 57 kD. The tetradecamer facilitates the in vitro folding of a number of proteins which would otherwise misfold or aggregate and precipitate. The structure of GroEL from E. coli has been established through X-ray crystallographic studies as reported by Braig K et al (1994) Nature 37l: 578-586. The holo protein is cylindrical, consisting of two seven-membered rings that form a large central cavity which according to Ellis R J and Hartl F U (1996) FASEB Journal 10: 20-26 is generally considered to be essential for activity. Some small proteins have been demonstrated to fold from their denatured states when bound to GroEL (Gray T E and Fersht A R (1993) J Mol Biol 232: 1197-1207; Hunt J F et al (1996) Nature 37: 3745; Weissman J S et al (1996) Cell 84: 481-490: Mayhew M et al (1996) Nature 379: 420426; Corrales F J and Fersht A R (1995) Proc Nat Acad Sci 92: 5326-5330) and it has been argued that a cage-like structure is necessary to sequester partly folded or assembled proteins (Ellis R J and Hartl F U (1996) supra.

The entire amino acid sequence of E. coli GroEL is also known (see Braig K et al (1994) supra) and three domains have been ascribed to each cpn60m of the holo chaperonin (tetradecamer). These are the intermediate (amino acid residues 1-5, 134-190, 377-408 and 524-548), equatorial (residues 6-133 and 409-523) and apical (residues 191-376) domains.

Monomers of GroEL have been induced by urea or pressure, but they are inactive and have to reassociate to form the central cavity in order to facilitate the refolding of rhodanese (Mendoza J A et al (1994) J Biol Chem 269: 2447-2451; Ybarra J and Horowitz P M (1995) J Biol Chem 27: 22962-22967).

GroEL facilitates the folding of a number of proteins by two mechanisms; (1) it prevents aggregation by binding to partly folded proteins (Goloubinoff P et al (1989) Nature 342: 884-889; Zahn R and Pluickthun A (1992) Biochemistry 21: 3249-3255), which then refold on GroEL to a native-like state (Zahn R and Plückthun A (1992) Biochemistry 21: 3249-3255; Gray T E and Fersht A R (1993) J Mol Biol L: 1197-1207); and (2) it continuously anneals misfolded proteins by unfolding them to a state from which refolding can start again (Zahn R et al (1996) Science 271: 642-645). Some mutations in the apical domain led to a decrease in polypeptide binding (Fenton W A et al (1994) Nature 371: 614-619), suggesting that this domain is involved in the binding of polypeptides. Electron microscopy suggests that denatured protein binds to the inner side of the apical end of the GroEL-cylinder (Chen S et al (1994) Nature 371: 261-264). The equatorial domain has been shown from the 2.4 Å crystal structure of ATPγS-ligated GroEL (Boisvert D C et al (1996) Nature Structure Biology 3: 170-177) and mutagenesis studies (Fenton W A et al (1994) Nature 371: 614-619) to have the nucleotide binding sites. Binding and hydrolysis of ATP is cooperative (Bochkareva E S et al (1992) J Biol Chem 267: 6796-6800; Gray T E and Fersht A R (1991) FEBS Lett 292: 254-258), and lowers the affinity for polypeptides (Jackson G S et al (1993) Biochemistry 32: 2554-2563). Most of the intermolecular contacts between the subunits of GroEL are between the equatorial domain. The intermediate domain connects the other two domains, transmitting allosteric effects (Braig K et al (1994) Nature 371: 578-586; Braig K et al (1995) Nature Struct Biol 2: 1083-1094).

The crystal structure of GroEL shows unusually high B-factors for the apical domain compared with the equatorial or intermediate domain, and the B-factors vary considerably within the domain (Braig K et al (1994) Nature 371: 578-586; Braig K et al (1995) Nature Struct Biol 2: 1083-1094; Boisvert D C et al (1996) Nature Structure Biology 3: 170-177). The high overall B-factor seems to result from a static disorder within the asymmetric unit and probably throughout the crystals of GroEL, and has been attributed to rigid-body movements generated by hinge-like β-sheets in the intermediate domain. Regions of high flexibility have also been observed in the 2.8 Å structure of the co-chaperonin GroES (Hunt J F et al (1996) Nature 379: 3745). A mobile loop has been shown to be directly involved in ADP-dependent binding to the apical domain (Landry S J et al (1993) Nature 36: 255-258). Binding of GroES leads to a conformational change of GroEL and a concomitant enlargement of the GroEL-cavity (Chen S et al (1994) Nature 371: 261-264), in which the encapsulated polypeptide substrate can refold to a native-like state without the danger of aggregation (Martin J et al (1993) Nature 366: 228-233; Weissman J S et al (1995) Cell 83: 577-587).

Monomeric forms of GroEL have been induced by site-directed mutagenesis and expressed and although these bind to rhodanese they do not affect its refolding (White Z W et al (1995) J Biol Chem 270: 20404-20409).

Yoshida et al (1993) FEBS I: 363-367 report that a 34 kD proteolytic fragment of E. coli GroEL which lacks 149 NH₁-terminal residues and ˜93 COOH-terminal residues (GroEL 150-456) facilitates refolding of denatured rhodanese in the absence of GroES and ATP. Although the proteolytic fragment GroEL 150-456 elutes as a monomer during gel filtration, it still comprises the apical domain and significant portions of the intermediate and equatorial domains, the latter of which determine the intersubunit contacts of GroEL (Braig K et al (1994) supra), thus allowing transient formation of the central cavity thereby accounting for the chaperonin activity which is observed.

In any event, the mode of rhodanese refolding by GroEL 150-456 is very different from that brought about by the holo protein; the yield of productive refolding is low, folding is rapidly saturated with time, and it is not affected by GroES and ATP. Efficient release and folding requires the hydrolysis of ATP (Landry S J et al (1992) Nature 355: 455457; Gray T E and Fersht A R (1992) FEBS Lett 282: 254-258; Jackson G S et al (1993) Biochemistry 32: 2554-2563; Todd M et al (1993) Biochemistry 32: 8560-8567.)

EP-A-0 650 975 (NIPPON OIL CO LTD) discloses chaperoin molecules and a method of refolding denatured proteins using GroEL chaperonin 60 monomers (cpn60 m) obtained from Thermus thermophilus. The holo-chaperonin was first extracted and then purified from the bacterial source according to the method of Taguchi et al (1991) J Biol Chem 2X: 22411-22418. The cpn60m was then produced by treatment of the holo-chaperonin with trifluoroacetic acid (TFA) followed by reverse phase (rp) HPLC of the resulting denatured protein. A peak fraction containing the approximately 57 kD cpn60m was obtained. The refolding activity of the cpn60 m was assayed in solution by monitoring the regain in activity of inactivated rhodanese, which in specific activity terms amounted to about only 25% of the specific activity of the rhodanese prior to inactivation. When background spontaneous rhodanese refolding is subtracted then there is only an approximately 20% refolding activity.

As well as cpn60m, EP-A-0 650 975 also discloses the use of an approximately 50 kD N-terminal deletion fragment of cpn60m wherein the N-terminal amino acid residues up to (but not including) the Thr residue at position 79 are removed by proteolysis. This 5OkD fragment showed an approximately 35% (about 30% when background is subtracted) rhodanese refolding activity when in solution.

Taguchi H et al (1994) J Biol Chem 269: 8529-8534 is a scientific report on which the invention of EP-A-0 650 975 is based. A transiently formed GroEL tetradecamer (the holo-chaperonin) was perceived to exist when the chaperonin monomers are present in solution. Consequently, the refolding activity of these preparations can be seen to be caused by the presence of holo chaperonin, not monomers. To test this, Taguchi et a! immobilised cpn60 m to a chromatographic resin to exclude the possibility of holo chaperonin formation. When immobilised and therefore when in truly monomeric form, cpn60 m exhibited only about 10% rhodanese refolding activity.

The refolding of rhodanese has been a common and convenient assay to determine chaperonin activity but it has been observed that there are significant problems with the assay which cast serious doubt on existing assertions of refolding activity based on this assay. The fact is that rhodanese refolds spontaneously in the absence of molecular chaperones with the yield of refolded rhodanese increasing progressively as the rhodanese concentration decreases (see Taguchi et al (1994) supra). The 10% of rhodanese refolding activity reported in EP-A-0 650 975 for immobilised (truly monomeric) cpn60m is therefore too close to the spontaneous regain of activity by rhodanese to demonstrate that any monomeric chaperonin has a refolding activity towards proteins generally, let alone cpn60m and rhodanese.

Alconada A and Cuezva J M (1993) TIBS 18: 81-82 suggested that an “internal fragment” of GroEL may possess a chaperone activity on the basis of amino acid sequence similarity between the altered trRNA stability (ams) gene product (Ams) of E. coli and the central part of GroEL. The ams locus is a temperature-sensitive mutation that maps at 23 min on the E. coli chromosome and results in mRNA with an increased half-life. The ams gene has been cloned, expressed and shown to complement the ams mutation. The gene product is a 149-arnino acid protein (Ams) with an apparent molecular weight of 17 kD.

Chanda P K et al (1985) J Bacteriol 161: 446-449 found that a 17 kD protein fragment corresponding to part of the L gene of the groE operon when expressed in E. coli ams mutants restores the wild-type phenotype. This 17kID fragment was suggested as being an isolated, functional chaperonin protein module. The amino acid sequences of three chaperonins (E. coli GroEL, ribulose bisphosphate carboxylase (RUBPC) subunit-binding protein from Triticum aestivum and Saccharomvces cerevisiae mitochondrial hsp60) were compared with the sequence of Ams. Residues 307423 were found to correspond substantially between Arns and GroEL. These residues comprise nearly equivalent portions of both the intermediate and apical domains of GroEL.

The sequence alignments of Ams protein with the chaperonins noted above reveals a striking similarity (98%) between the amino-terminal four-fifths of Ams and the central part (approximately one-fifth) of E. coli GroEL chaperonin. The 50% sequence similarity between the Ams amino terminal region and the two other chaperonins is in line with the reported identity among the chaperonin family. The carboxy-terminal part of the Ams protein showed no similarity with -chaperonins (<10% homology).

The present inventors have identified a need for a simple, truly monomeric chaperone molecule which is of defined sequence and structure and which can efficiently and reproducibly refold, renature, reactivate or recondition proteins from a range of sources in the absence of added cofactor or other agents and without the need to associate to form a holo chaperonin. A problem which the invention seeks to solve is therefore how to provide an active portion or fragment of a chaperone in truly monomeric form so as to promote a useful and efficient reagent for the refolding, renaturing or reconditioning of biological molecules, particularly proteins. A further object is the provision of such a monomeric form at minimum size.

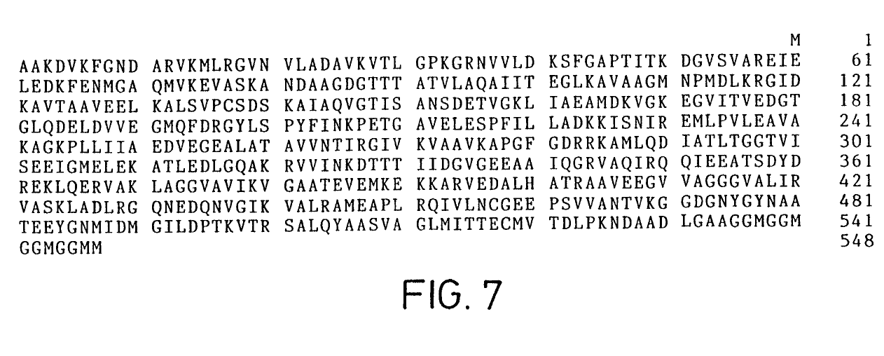

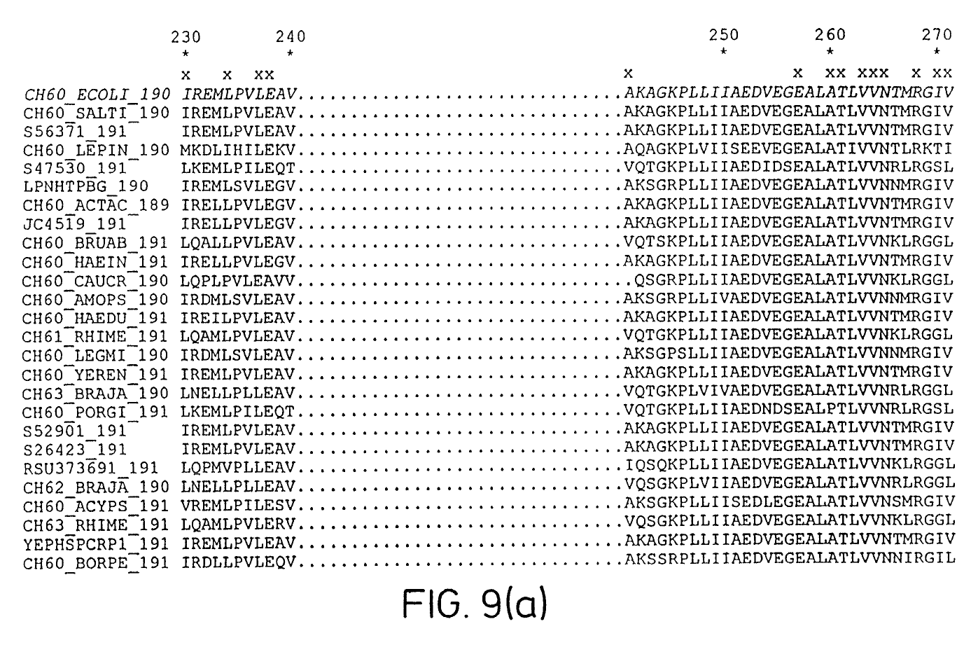

In first aspect the present invention provides a chaperone polypeptide having an amino acid sequence selected from at least amino acid residues 230-271 but no more than residues 150455 or 151456 of a GroEL sequence substantially as shown in FIG. 7, or a corresponding sequence of a substantially homologous chaperone polypeptide, or a modified, mutated or variant thereof having chaperone activity.

The sequence of GroEL is available in the art, as set forth above, and from academic databases; however, GroEL fragments which conform to the database sequence are inoperative. Specifically, the database contains a sequence in which positions 262 and 267 are occupied by Alanine and Isoleucine respectively. Fragments incorporating one or both of these residues at these positions are inoperative and unable to promote the folding of polypeptides. The invention, instead, relates to a GroEL polypeptide in which at least one of positions 262 and 267 is occupied by Leucine and Methionine respectively.

The amino acid sequence is preferably selected from at least amino acid residues 193-335, preferably 193-337, more preferably 191-345, even more preferably 191-376 but no more than residues 151-455. The invention therefore includes polypeprides being GroEL amino acid residues 230-271, 230-272 . . . et seq . . . 230-455 and in like manner residues 230-271, 229-271 . . . et seq . . . 151-271. Also, residues 230-271, 229-272 . . . et seq . . . 151-351, 151-352 . . . et seq . . . 151-455. All amino acid sequences of 42 or more residues comprising at least contiguous residues 230-271 and not exceeding 151455 are within the scope of this aspect of the invention eg 171-423 or 166-406.

In a highly preferred aspect, the invention provides fragments selected from the group consiting of residues 191-375, 191-345 and 193-335.

There are four key properties that may characterise a protein as a molecular chaperone (1) suppression of aggregation during protein folding; (2) suppression of aggregation during protein unfolding; (3) influence on the yield and kinetics of folding; and (4) effects exerted at near stoichiometric levels.

Chaperone activity may be determined in practice by an ability to refold cyclophilin A but other suitable proteins such as glucosamine-6-phosphate deaminase or a mutant form of indoleglycerol phosphate synthase (IGPS) (amino acid residues 49-252) may be used. A rhodanese refolding assay may also be used. Details of suitable refolding assays are described in more detail in the specific examples provided hereinafter.

In second aspect the invention provides monomeric polypeptide having chaperone activity and incapable of multimerisation in solution.

In third aspect the invention provides a chaperone polypeptide which, when in solution, remains monomeric and has the ability to refold, reactivate or recondition proteins, said polypeptide including the protein binding active site motif:

1 X X X 2 X X 3 4 X X 5 X X X X X X X X X X X X X X X 6 X X 7 8 X 9 10 X X 12 X 13 14

wherein 1 is selected from amino acid residues:

I, M, L, V, S, For A;

wherein 2 is selected from: L,I,P,VorA;

wherein 3 is selected from: L, E, V, H or I;

wherein 4 is selected from: E, A, R, L, Q, or N;

wherein 5 is selected from: A, V, I, M, L, N, S, R, T, Q or K;

wherein 6 is selected from: E, D or G;

wherein 7 is selected from: A, P, S, T, G or L;

wherein 8 is selected from: T, A, N, S or V;

wherein 9 is selected from: V, L, I or A;

wherein 10 is selected from: V, L, I, F or H;

wherein 11 is selected from: N, S or L;

wherein 12 is selected from: R, K, N, Q, L or S;

wherein 13 is selected from: I, T, S, G, V, A, Q, N, K, F or P;

wherein 14 is selected from: V, I, L, F, D or T; and

wherein the X's represent a peptide bond or bonds or at least one amino acid residue, or a functional variant thereof in which one or more of the numbered amino acid residues 1 to 14 has undergone a conservative substitution.

In fourth aspect the invention provides a chaperone polypeptide which, when in solution, remains monomeric and has the ability to refold, reactivate or recondition proteins, said polypeptide including at least one protein binding active site motif moiety selected from:

(a) 1 X X X 2 X X 3 4 X and

(b) X 5X X X X X X X X X X X X X X X 6 X X 7 8 X 9 10 11 X X 12 X 13 14

wherein 1 is selected from amino acid residues:

I, M, L, V, S, F or A;

wherein 2 is selected from: L, I, P, VorA;

wherein 3 is selected from: L, E, V, H or I;

wherein 4 is selected from: E, A, R, L, Q, or N;

wherein 5 is selected from: A, V, I, M, L, N, S, R, T, Q or K;

wherein 6 is selected from: E, D or G;

wherein 7 is selected from: A, P, S, T, G or L;

wherein 8 is selected from: T, A, N, S or V;

wherein 9 is selected from: V, L, I or A;

wherein 10 is selected from: V, L, I, For H;

wherein 11 is selected from: N, S or L;

wherein 12 is selected from: R, K, N, Q, L or S;

wherein 13 is selected from: I, T, S, G, V, A, Q, N, K, F or P;

wherein 14 is selected from: V, I, L, F, D or T; and

wherein X is at least one amino acid residue, or a functional variant thereof in which one or more of the numbered amino acid residues 1 to 14 has undergone a conservative substitution.

In fifth aspect the claim provides a chaperone polypeptide which, when in solution, remains monomeric and has the ability to refold, reactivate or recondition proteins, said polypeptide including the protein binding active site motif:

1 X X X L X X L E X X A X X X X X X X X X X X X X X X E X X A T X V V N X X R X I V

wherein X is at least one amino acid residue, or a functional variant thereof in which one or more of the specified amino acid residues has undergone a conservative substitution.

In sixth aspect the claims provides a chaperone polypeptide which, when in solution, remains monomeric and has the ability to refold, reactivate or recondition proteins, said polypeptide including at least one protein binding active site motif moiety selected from:

(a) I X X X L X X L E X

(b) X A X X X X X X X X X X X X X X X E X X A T X V V N X X R X I V

wherein X is at least one amino acid residue, or a functional variant thereof in which one or more of the specified amino acid residues has undergone a conservative substitution.

A conservative substitution is the replacement of one amino acid residue for another chemically or functionally similar amino acid residue such that the function of the polypeptide overall remains substantially unchanged.

The terms “refold”, “reactivate” and “recondition” are not intended as being mutually exclusive. For example, an inactive protein, perhaps denatured using urea may have an unfolded structure. This inactive protein may then be refolded with a polypeptide of the invention thereby reactivating it. In some circumstances there may be an increase in the specific activity of the refolded/reactivated protein compared to the protein prior to inactivation/denaturation: this is termed “reconditional”.

Preferably, the active site motif or an active site motif moiety includes the conserved sequence:

PLL(V)I(V)IA(S)EDV(I)EGEAL

in which amino acid symbols in parenthesis are alternatives to the immediately preceding symbol reading left to right.

In seventh aspect the invention provides monomeric polypeptide having chaperone activity and incapable of multimerisation characterised in that in the absence of ATP the polypeptide has a protein refolding activity of more than 50%, preferably 60%, even more preferably 75%, said refolding activity being determined by contacting the polypeptide with an inactivated protein of known specific activity prior to inactivation. and then determining the specific activity of the said protein after contact with the polypeptide. the % refolding activity being:

$$\frac{specific activity of protein after contact with polypeptide}{specific activity of protein prior to inactivation} \times \frac{100}{1}$$

Preferably, the chaperone activity is determined by the refolding of cyclophilin A. More preferably, 8M urea denatured cyclophilin A (100 μM) is diluted into 100 mM potassium phosphate buffer pH7.0, 10 mM DTT to a final concentration of 1 μM and then contacted with at least 1 μM of said polypeptide at 25° C. for at least 5 min, the resultant cyclophilin A activity being assayed by the method of Fischer G et al (1984) Biomed Biochim Acta 43: 1101-1111.

The polypeptide is preferably an hsp60 polypeptide, preferably a GroEL polypeptide.

In eighth aspect the invention provides a polypeptide as claimed in any preceding claim which comprises at least an amino acid sequence selected from GroEL residues:

(a) 191-329, 191-330, 191-331, 191-332, 191-333, 191-334, 191-335, 191-336, 191-337, 191-338, 191-339, 191-340, 191-341, 191-342, 191-343, 191-344, 191-345, 191-346, 191-347, 191-348, 191-349, 191-350, 191-351, 191-352, 191-353, 191-354, 191-355, 191-356, 191-357, 191-358, 191-359, 191-360, 191-361, 191-362, 191-363, 191-364, 191-365, 191-366, 191-367, 191-368, 191-369, 191-370, 191-371, 191-372, 191-373, 191-374, 191-375 or 191-376, or

(b) 192-329, 192-330, 192-331, 192-332, 192-333, 192-334, 192-335, 192-336, 192-337, 192-338, 192-339, 192-340, 192-341, 192-342, 192-343, 192-344, 192-345, 192-346, 192-347, 192-348, 192-349, 192-350, 192-351, 192-352, 192-353, 192-354, 192-355, 192-356, 192-357, 192-358, 192-359, 192-360, 192-361, 192-362, 192-363, 192-364, 192-365, 192-366, 192-367, 192-368, 192-369, 192-370, 192-371, 192-372, 192-373, 192-374, 192-375 or 192-376. or

(c) 193-329, 193-330, 193-331, 193-332, 193-333, 193-334, 193-335, 193-336, 193-337, 193-338, 193-339, 193-340, 193-341, 193-342, 193-343, 193-344, 193-345, 193-346, 193-347, 193-348, 193-349, 193-350, 193-351, 193-352, 193-353, 193-354, 193-355, 193-356, 193-357, 193-358, 193-359, 193-360, 193-361, 193-362, 193-363, 193-364, 193-365, 193-366, 193-367, 193-368, 193-369. 193-370, 193-371, 193-372, 193-373, 193-374, 193-375 or 193-376, or

(d) 230-271. 229-271, 229-272, 228-272, 228-273, . . . et seq . . . 194-328, 194-329, or

the equivalent residues of substantially homologous chaperoning, or a modified, mutated or variant sequence thereof.

A preferred polypeptide has the amino acid sequence 191-345 or 191-376, more preferably 193-335 or 191-337 of GroEL, or the equivalent residues of substantially homologous chaperoning, or a modified, mutated or variant sequence thereof.

The polypeptide preferably has a molecular weight of less than 34 kDa.

“Modifications” include chemically modified polypeptides for example. “Variants” include, for example, naturally occurring variants of the kind to be found amongst a population of hsp60 chaperonin harbouring organisms/cells as well as naturally occurring polymorphisms or mutations. “Mutations” may also be introduced artificially by processes of mutagenesis well known to a person skilled in the art.

In being “substantially homologous” peptides may have at least 50% amino acid sequence homology with the specified GroEL amino acid sequences, preferably at least 60% homology and more preferably 75% homology. Homology may of course also reside in the nucleotide sequences for the polypeptide which may be at least 50%, preferably at least 60% homologous and more preferably 75% homologous with the nucleotide sequence encoding the specified GroEL amino acid residues.

The hsp60 class of chaperonin proteins are generally homologous in structure and so there are therefore conserved or substantially homologous amino acid sequences between the members of the class. GroEL is just an example of an hsp60 chaperonin protein; other suitable proteins having an homologous apical domain may be followed.

The list was compiled from the OWL database release 28.1. The sequences listed below show clear homology to apical domain (residues 191-375) in PDB structure pdblgrl.ent.

OWL is a non redundant database merging SWISS-PROT, PIR (1-3), GenBank (translation) and NRL-3D.

190-374 CH60_ECOLI 60 KD CHAPERONIN (PROTEIN CPN60)(GROEL PROTEIN)(AMS).—ESCHERICHIA 190-374 CH60_SALTI 60 KD CHAPERONIN (PROTEIN CPN60)(GROEL PROTEIN).—SALMONELLA TYPHI. 191-375 S56371 GroEL protein—Escherichia coli 190-374 CH60_LEPIN 60 KD CHAPERONIN (PROTEIN CPN60)(GROEL PROTEIN)(HEAT SHOCK 58 KD PRO 191-375 S47530 groEL protein—Porphyromonas gingivalis 190-374 LPNHTPBG LPNHTPBG NID:gl49691—Legionella pneumophila (strain SVir)(library- 189-373 CH60_ACTAC 60 KD CHAPERONIN (PROTEIN CPN60)(GROEL PROTEIN).—ACTINOBACILLUS ACT 191-375 JC4519 heat-shock protein GroEL—Pasteurella multocida

191-375 CH60BRUAB 60 KD CHAPERONIN (PROTEIN CPN60)(GROEL PROTEIN).—BRUCELLA ABORTUS. 191-375 CH60_HAEIN 60 KD CHAPERONIN (PROTEIN CPN60)(GROEL PROTEIN).- HAEMOPHILUS INFLUE 190-373 CH60_CAUCR 60 KD CHAPERONIN (PROTEIN CPN60)(GROEL PROTEIN).—CAULOBACTER CRESCE 190-374 CH60_AMOPS 60 KD CHAPERONIN (PROTEIN CPN60)(GROEL PROTEIN).—AMOEBA PROTEUS SYM 191-375 CH60_HAEDU 60 KD CHAPERONIN (PROTEIN CPN60)(GROEL PROTEIN).—HAEMOPHILUS DUCREY 191-375 CH61_RHIME 60 KD CHAPERONIN A (PROTEIN CPN60 A)(GROEL PROTEIN A).—RHIZOBIUM ME 190-374 CH60_LEGMI 60 KD CHAPERONIN (PROTEIN CPN60)(GROEL PROTEIN)(58 KD COMMON ANTIGEN 191-375 CH60_YEREN 60 KD CHAPERONIN (PROTEIN CPN60) (GROEL PROTEIN) (HEAT SHOCK PROTEIN 6) 190-374 CH 63_BRAJA 60 KD CHAPERONIN 3 (PROTEIN CPN60 3)(GROEL PROTEIN 3).—BRADYRHIZOBI 191-375 CH60_PORGI 60 KD CHAPERONIN (PROTEIN CPN60)(GROEL PROTEIN).—PORPHYROMONAS GING 191-375 S52901 heat shock protein 60K—Yersinia enterocolitica 191-375 S26423 heat shock protein 60 —Yersinia enterocolitica

191-375 RSU373691 RSU37369 NID: gl208541—Rhodobacter sphaeroides strain=HR. 190-374 CH62_BRAJA 60 KD CHAPERONIN 2(PROTEIN CPN60 2)(GROEL PROTEIN 2). —BRADYRHIZOBI 191-375 CH60_ACYPS 60 KD CHAPERONIN (PROTEIN CPN60)(GROEL PROTEIN)(SYMBIONIN).—ACYRTH 191-375 CH63_RHIME 60 KD CHAPERONIN C(PROTEIN CPN60 C)(GROEL PROTEIN C).—RHIZOBIUM ME 191-375 YEPHSPCRP1 YEPHSPCRP NID: g466575—Yersinia enterocolitica DNA. 191-375 CH60_BORPE 60 KD CHAPERONIN (PROTEIN CPN60)(GROEL PROTEIN).—BORDETELLA PERTUSS 189-373 BRUGRO1 BRUGRO NID: gl44106—Brucella aabortus (library: lambda-2001) DNA.

191-375 CH60_PSEAE 60 KD CHAPERONIN (PROTEIN CPN60)(GROEL PROTEIN).—PSEUDOMONAS AERUGI 190-374 CH60_BARBA 60 KD CHAPERONIN (PROTEIN CPN60)(IMMUNOREACTIVE PROTEIN BB65)(IMMUNO 191-375 BAOBB63A BAOBB63A NID: gl43845 -Bartonella bacilliformis (library: ATCC 35685) 189-373 CH60_BACST 60 KD CHAPERONIN (PROTEIN CPN60)(GROEL PROTEIN).—BACILLUS STEAROTHE 188-372 190-373 CH60_BORBU 60 KD CHAPERONIN (PROTEIN CPN60)(GROEL PROTEIN).—BORRELIA BURGDORFE 224-408 S26583 chaperonin hsp60—maize 190-373 A49209 heat shock protein HSP60 -Lyme disease spirochete 224-408 MZECPN60B MZECPN60B NID: g309558—Zea mays (strain B73)(library:Dashll of P. S 189-373 CH60-THEP3 60 KD CHAPERONIN (PROTEIN CPN60)(GROEL PROTEIN)(HEAT SHOCK 61 KD PRO 188-372 .CH60_STAEP 60 KD CHAPERONIN (PROTEIN CPN60) (GROEL PROTEIN) (HEAT SHOCK PROTEIN 6 189-373 CH60_LACLA 60 KD CHAPERONIN (PROTEIN CPN60)(GROEL PROTEIN).—LACTOCOCCUS LACTIS 188-374 CH61_STRA 60 KD CHAPERONIN 1 (PROTEIN CPN60 1)(GROEL PROTEIN 1)(HSP58).—STRE 191-375 CH60_CHLPN 60 KD CHAPERONIN (PROTEIN CPN60)(GROEL PROTEIN).—CHLAMYDIA PNEUMONI 224-408 MZECPN60A MZECPN60A NID: g309556—Zea mays (strain 1373)(library:Dach 11 of P. 190-373 HECHSPAB1 HECHSPAB NID: g712829—Helicobacter pylori (individual_isolate 85P) D 221-405 CH60_ARATH MITOC1ONDRIAL CHAPERONIN HSP60 PRECURSOR.—ARABIDOPSIS THALIANA (MOUS 224-408 CHGO6MAIZE MITOCHONDRIAL CHAPERONIN HSP60 PRECURSOR.—ZEA MAYS (MAIZE). 190-374 CH60_CHLTR 60 KD CHAPERONIN (PROTEIN CPN60)(GROEL PROTEIN)(57 KD CHLAMYDIAL HYP 189-373 CH60_STAAU 60 KD CHAPERONIN (PROTEIN CPN60)(GROEL PROTEIN) (HEAT SHOCK PROTEIN 6 189-373 CH60_CLOPE 60 KD CHAPERONIN (PROTEIN CPN60)(GROEL PROTEIN).—CLOSTRIDIUM PERFRI 212-397 HS60_YEAST HEAT SHOCK PROTEIN 60 PRECURSOR (STIMULATOR FACTOR 1 66 KD COMPONENT) 217-403 CH60_PYRSA 60 KD CHAPERONIN (PROTEIN CPN60)(GROEL PROTEIN).—PYRENOMONAS SALINA 191-377 CH60_EHRCH 60 KD CHAPERONIN (PROTEIN CPN60)(GROEL PROTEIN).—EHRLICHIA CHAFFEEN 191-375 CHTGROEl CHTGROE NID: gl44503—C. trachomatis DNA. 188-372 CH60 THETH, 60 KD CHAPERONIN(PROTEIN CPN60)(GROEL PROTEIN).—THERMUS AQUATICUS 189-373 TAU294831 TAU29483 NID: gll22940—Thermus acuaticus. 190-378 CH60_RICTS 60 KD CHAPERONIN (PROTEIN CPN60)(GROEL PROTEIN)(MAJOR ANTIGEN 58)(5 189-375 SYCCPNC SYCCPNC NID: g1001102 -Synechocystis sp. (strain PCC6803,) DNA.

190-373 CPU308211 CPU30821 NID: glOl6083—Cyanophora paradoxa. 189-373 CH61_MYCLE 60 KD CHAPERONIN 1 (PROTEIN CPN60 1)(GROEL PROTEIN 1).—MYCOBACTERIU 239-423 PSU21139 PSU21139 NID: g806807—pea. 191-377 CH60_COWRU 60 KD CHAPERONIN (PROTEIN CPN60)(GROEL PROTEIN).—COWDRIA RUMINANTIU 245-429 RUBB_BRANA RUBISCO SUBUNIT BINDING-PROTEIN BETA SUBUNIT PRECURSOR (60 KD CHAPERON 144-328 SCCPN60 SCCPN60 NID: gll67857—rye.

153-338 CH60_EHRRI 60 KD CHAPERONIN (PROTEIN CPN60)(GROEL PROTEIN)(55 KD MAJOR ANTIGEN) 245-429 RUBB-ARATH RUBISCO SUBUNIT BINDING-PROTEIN BETA.SUBUNIT PRECURSOR (60 KD CHAPERON 235-419 ATU49357 ATU49357 NID: gl223909—thale cress strain=ecotype Wassilewskija. 195-379 RUBI_BRANA RUBISCO SUBUNIT BINDING-PROTEIN ALPHA SUBUNIT (60 KD CHAPERONIN ALPHA 189-374 CH62-SYNY3 60 KD CHAPERONIN 2 (PROTEIN CPN60 2)(GROEL HOMOLOG 2).—SYNECHOCYSTI 178-362 RUBA_RICCO RUBISCO SUBUNIT BINDING-PROTEIN ALPHA SUBUNIT (60 KD CHAPERONIN ALPHA 190-375 CH60_ODOSI 60 KD CHAPERONIN (PROTEIN CPN60)(GROEL PROTEIN).—ODONTELLA SINENSIS 236-420 PSU21105 PSU21105 NID: gll85389—pea. 224-409 CH60_BRANA MITOCHONDRIAL CHAPERONIN CH60_BACSU 60 KD CHAPERONIN (PROTEIN CPN60)(GROEL PROTEIN).—BACILLUS SUBTILIS. 191-375 CH60_AGRTU 60 KD CHAPERONIN (PROTEIN CPN60)(GROEL PROTEIN).—AGROBACTERIUM TUME 191-375 b36917 heat shock protein GroEL—Agrobacterium tumefaciens

191-375 PAU17072 PAU17072 NID: g576778—Pseudomonas aeruginosa. 191-375 CH60 RHILV 60 KD CHAPERONIN (PROTEIN CPN60)(GROEL PROTEIN).—RHIZOBIUM LEGUMINO 187-373 CH61_STRCO 60 KD CHAPERONIN 1 (PROTEIN CPN60 1)(GROEL PROTEIN 1) (HSP58) .—STRE 191-375 CH60 COXBU 60 KD CHAPERONIN (PROTEIN CPN60) (GROEL PROTEIN) (HEAT SHOCK PROTEIN B 191-375 CH62_RHIME 60 KD CHAPERONIN B (PROTEIN CPN60 B)(GROEL PROTEIN B).—RHIZOBIUM ME 191-375 PSEGROESLI PSEGROESL NID: gl51241—Pseudomonas aeruginosa (library: ATCC 27853) 189-372 CH61_SYNY3 60 KD CHAPERONIN 1 (PROTEIN CPN60 1)(GROEL HOMOLOG 1).—SYNECHOCYSTI 189-373 CH6C_CLOTM 60 KD CHAPERONIN (PROTEIN CPN60)(GROEL PROTEIN)(HSP-60).—CLOSTRIDI 191-373 CH60_PSEPU 60 KD CHAPERONIN (PROTEIN CPN60)(GROEL PROTEIN).—PSEUDOMONAS PUTIDA 190-373 CH60_SYNP7 60 KD CHAPERONIN (PROTEIN CPN60)(GROEL PROTEIN).—SYNECHOCOCCUS SP. 190-374 CH60_GALSU 60 KD CHAPERONIN (PROTEIN CPN60)(GROEL PROTEIN).—GALDIERIA SULPHURA 190-374 CH60_ZYMMO 60 KD CHAPERONIN (PROTEIN CPN60)(GROEL PROTEIN).—ZYMOMONAS MOBILIS. 191-375 JC2564 heat shock protein groEL—Zymomonas mobilis

191-375 CH60 CHRVI 60 KD CHAPERONIN (PROTEIN CPN60)(GROEL PROTEIN).—CHROMATIUM VINOSUM 189-373 CH60_MYCTU 60 KD CHAPERONIN (PROTEIN CPN60)(GROEL PROTEIN)(65 KD ANTIGEN)(HEAT 191-375 CH60_NEIME 60 KD CHAPERONIN (PROTEIN CPN60)(GROEL PROTEIN)(63 KD STRESS PROTEIN 189-373 CH60 TREPA 60 KD CHAPERONIN (PROTEIN CPN60)(GROEL PROTEIN) (TPN60)(TP4 ANTIGEN) 190-374 CH60 HELPY 60 KD CHAPERONIN (PROTEIN CPN60) (GROEL PROTEIN) (HEAT SHOCK PROTEIN 6 191-375 CH60_NEIGO 60 KD CHAPERONIN (PROTEIN CPN60)(GROEL PROTEIN)(63 KD STRESS PROTEIN 222-406 CH61_CUCMA MITOCHONDRIAL CHAPERONIN HSP60-1 PRECURSOR. —CUCURBITA MAXIMA (PUMPKI 189-373 CH60 MYCPA 60 KD CHAPERONIN (PROTEIN CPN60)(GROEL PROTEIN)(65 KD ANTIGEN)(HEAT 230-414 MPU15989 MPU15989 NID:g559802 -Mycobacterium paratuberculosis. 224-408 S26582 chaperonin hsp60—maize 191-375 S40247 heat-shock protein—Neisseria gonorrhoeae 189-373 CH60_CLOAB 60 KD CHAPERONIN (PROTEIN CPN60)(GROEL PROTEIN).—CLOSTRIDIUM ACETOB 191-375 CH60_NEIFL 60 KD CHAPERONIN (PROTEIN CPN60)(GROEL PROTEIN)(63 KD STRESS PROTEIN 190-373 CH60_LEGPN 60 KD CHAPERONIN (PROTEIN CPN60)(GROEL PROTEIN)(58 KD COMMON ANTIGEN 222-406 CH62_CUCMA MITOCHONDRIAL CHAPERONIN HSP60-2 PRECURSOR.—CUCURBITA MAXIMA (PUMPKI 191-375 CHTGROESL1 CHTGROESL NID: g402332—Chlamydia trachomatis DNA. 64-248 S40172 S40172 NID: g251679—Chlamydia psittaci pigeon strain P-1041. 189-373 SYOGROEL2 SYOGROEL2 NID:g562270—Synechococcus vulcanus DNA. 191-375 CH60_CHLPS 60 KD CHAPERONIN (PROTEIN CPN60)(GROEL PROTEIN)(57 KD CHLAMYDIAL HYP 188-372 CH62_STRAL 60 KD CHAPERONIN 2 (PROTEIN CPN60 2)(GROEL PROTEIN 2)(HSP56).—STRE 189-373 CH62_MYCLE 60 KD CHAPERONIN 2 (PROTEIN CPN60 2)(GROEL PROTEIN 2)(65 KD ANTIGEN) 236-420 MSGANTM MSGANTM NID: gl49923—M.leprae DNA, clone Y3178.

CPN60 PRECURSOR.—BRASSICA NAPUS (RAPE). 105-289 PMSARG2 PMSARG2 NID: g607157—Prochlorococcus marinus. 234-417 RUB2_BRANA RUBISCO SUBUNIT BINDING-PROTEIN ALPHA SUBUNIT PRECURSOR (60 KD CHAPERO 75-259 CRECPNlA CRECPN1A NID: g603910—Chlamydomonas reinhardtii cDNA to mRNA. 215-400 P60_CRIGR MITOCHONIDRIAL MATRIX PROTEIN P1 PRECURSOR (P60 LYMPHOCYTE PROTEIN)(CH224-408 CRECPN1B CRECPN1B NID: g603912—Chlamydomonas reinhardtii cDNA to mRNA. 191-375 RUBA WHEAT RUBISCO SUBUNIT BINDING-PROTEIN ALPHA SUBUNIT PRECURSOR (60 KD CHAPERO 189-373 B47292 heat shock protein groEL—Mycobacterium tuberculosis 206-391 CELHSP60CP CELHSP60CP NID: g533166—Caenorhabditis elegans (strain CB1392) cDNA 215-400 P60_HUMAN MITOCHONDRIAL MATRIX PROTEIN P1 PRECURSOR (P60 LYMPHOCYTE PROTEIN)(CH 215-400 P60_MOUSE MITOCHONDRIAL MATRIX PROTEIN P1 PRECURSOR (P60 LYMPHOCYTE PROTEIN)(CH 215-400 P60_RAT MITOCHONDRIAL MATRIX PROTEIN P1 PRECURSOR (P60 LYMPHOCYTE PROTEIN)(CH 215-400 A41931 chaperonin hsp60—mouse

197-382 MMHSP60A MMHSP60A NID:g5l451—house mouse. 218-402 CH63_HELVI 63 KD CHAPERONIN PRECURSOR (P63).—HELIOTHIS VIRESCENS (NOCTUID MOTH) 205-390 EGHSP60GN EGHSP60GN NID: gl217625—Euglena gracilis. 222-407 HS60_SCHPO PROBABLE HEAT SHOCK PROTEIN 60 PRECURSOR.—SCHIZOSACCHAROMYCES POMBE 198-385 S61295 heat shock protein 60—Trypanosoma cruzi

198-385 TRBMTHSP TRBMTHSP NID: g903883—Mitochondrion Trypanosoma brucei (strain EATRO 8-69 ECOGROELA ECOGROELA NID: gl46268—E. coli DNA, clone E. 142-325 ENHCPN60P ENHCPN60P NID: g675513—Entamoeba histolytica (strain HM-1:IMSS) DNA. 257-433 CH60_PLAFG MITOCHONDRIAL CHAPERONIN CPN60 PRECURSOR.—PLASMODIUM FALCIPARUM (ISO 1-90 CRECPN1C CRECPN1C NID: g603914—Chlamydomonas reinhardtii cDNA to mRNA.

5-65 ATTS0779 ATTS0779 NID: gl7503—thale cress. 189-373 CH60 MYCGE 60 KD CHAPERONIN (PROTEIN CPN60)(GROEL PROTEIN).—MYCOPLASMA GENITAL 228-411 HTOHSP60X HTOHSP60X NID: g553068—Histoplasma capsulatum (strain G217B) DNA. 190-297 CH60 SYNP6 60 KD CHAPERONIN (PROTEIN CPN60)(GROEL PROTEIN)(FRAGMENT).—SYNECHO 169-245 RU3A_ARATH RUBISCO SUBUNIT BINDING-PROTEIN ALPHA SUBUNIT (60 KD CHAPERONIN ALPHA

The polypeptide may further comprise a polyamino acid sequence, preferably an N-terminal polyamino acid sequence although a C-terminal sequence may be present instead or in addition to an N-terminal sequence. The polyamino acid sequence may be selected from the same or different amino acid residues. When the same amino acid residue is repeated a particularly preferred polyamino acid sequence is a polyhistidine sequence.

Whether composed of the same or different amino acid residues, the further polyamino acid sequence may comprise any number of amino acid residues so long as chaperonin activity is provided. The other amino acid residues which may be included in the further polyamino acid sequence could be selected from any of the twenty or so essential amino acids common to biological systems or of any amino acid variant or derivative. When the polyamino acid sequence is other than a homopolymer then it may comprise a repeated sequence of two or more amino acid residues. The amino acid sequence may encode a portion of another known protein or polypeptide or it may even be random.

The further polyamino acid sequence preferably also includes a cleavage site cleavable by a cleavace agent. A preferred cleavage agent is thrombin although any other suitable agent will suffice. The sequence of amino acid residues is of course selected to permit cleavage by the desired cleavage agent.

The further polyamino acid sequence preferably comprises 17 to 39 amino acids although more than 39 and less than 17 amino acids may also be employed.

The polypeptide may be attached to a support, preferably in immobilised form, optionally immobilised to a chromatographic matrix. more preferably an agarose resin. When an agarose resin is used it is preferably a nickel-nitrilo-tri-acetic acid (NTA)-ligated agarose resin. This has affinity for a polypeptide having a polyhistidine tail.

The polypeptide of the invention may be obtained by recombinant means. Alternatively, the polypeptide may be produced by a routine chemical synthesis using standard polypeptide synthesis procedures known in the art. If produced by recombinant means the polypeptide may be fused to a heterologous protein or polypeptide.

The inventors have found that the fragments with polyhistidine tails of 17 amino acid residues sht-GroEL 193-335, sht-GroEL191-345 and sht-GroEL191-376 as well as GroEL191-345 are poteint facilitators of the folding of inactive forms of cyclophilin A and rhodanese, and catalyse the unfolding of barnase. Maximal refolding yield was obtained at stoichiometric concentrations of cyclophilin A and apical domain, indicating the formation of a 1:1 complex between chaperone fragment and substrate protein during refolding. The fragments sht-GroEL 193-335, sht-GroEL191-345 and sht-GroEL191-376 when attached to nickel-nitriol-tri-acetic acid (NTA)- ligated agarose resin or NHS agarose resin are also active in enhancing the folding yield of destabilised mutants of indole glycerol-phosphate synthase and of the protein cyclophilin A. This proves the monomer has refolding activity and also demonstrates that the imnobilised protein is functionally useful.

Fragments of GroEL may be produced either from recombinant DNA methods or from protein chemistry or any means of chemical synthesis, or the equivalent fragments of homologous hspα(cpn60) proteins or any natural variants or any variants produced by mutagenesis or of larger fragments containing the sequences 191-376, 191-345 or 193-335 of GroEL from E. coli.

In tenth aspect the invention provides a nucleic acid molecule comprising a nucleotide sequence encoding a polypeptide of the invention, or a nucleotide sequence hybridisable thereto and optionally encoding a polypeptide having chaperone activity.

Preferred characteristics of the nucleotide sequence correspond to the preferred features of the polypeptides of the aspects of the invention as hereinbefore defined.

This aspect of the invention therefore includes a recombinant DNA molecule for use in cloning and/or expressing a DNA sequence, said recombinant DNA molecule comprising:

(a) a nucleotide sequence encoding amino acid residues 191-376 of GroEL,

(b) a nucleotide sequence encoding amino acid residues 191-345 of GroEL,

(c) a nucleotide sequence encoding amino acid residues 193-337 of GroEL,

(d) a nucleotide sequence encoding amino acid residues 193-335 of groEL,

(e) a nucleotide sequence encoding amino acid residues of GroEL selected from amino acid residues:

(i) 191-329, 191-330, 191-331, 191-332, 191-333, 191-334, 191-335. 191-336, 191-337, 191-338, 191-339, 191-340, 191-341, 191-342, 191-343, 191-344, 191-345, 191-346, 191-347, 191-348, 191-349, 191-350, 191-351, 191-352, 191-353, 191-354, 191-355, 191-356, 191-357, 191-358, 191-359, 191-360, 191-361, 191-362, 191-363, 191-364, 191-365, 191-366, 191-367, 191-368, 191-369, 191-370, 191-371, 191-372, 191-373, 191-374, 191-375 or 191-376, or

(ii) 192-329, 192-330, 192-331, 192-332, 192-333, 192-334, 192-335, 192-336, 192-337, 192-338, 192-339, 192-340, 192-341, 192-342, 192-343, 192-344, 192-345, 192-346, 192-347, 192-348, 192-349, 192-350, 192-351, 192-352, 192-353, 192-354, 192-355, 192-356, 192-357, 192-358, 192-359, 192-360, 192-361, 192-362, 192-363, 192-364, 192-365, 192-366, 192-367, 192-368, 192-369, 192-370, 192-371, 192-372, 192-373, 192-374, i92-375 or 192-376. or

(iii) 193-329, 193-330, 193-331, 193-332, 193-333, 193-334, 193-335, 193-336, 193-337, 193-338, 193-339, 193-340, 193-341, 193-342, 193-343, 193-344, 193-345, 193-346, 193-347, 193-348, 193-349, 193-350, 193-351, 193-352, 193-353, 193-354, 193-355, 193-356, 193-357, 193-358, 193-359, 193-360, 193-361, 193-362, 193-363, 193-364, 193-365, 193-366, 193-367, 193-368, 193-369, 193-370, 193-371, 193-372, 193-373, 193-374, 193-375 or 193-376.

(f) 230-271, 229-271, 229-272, 228-272, 228-273, . . . et seq . . . 194-328, 194-329, or

(g) a nucleotide sequence hybridisable to any of (a), (b), (c), (d), (e) or (f) above and encoding a monomeric polypeptide having chaperone activity, or

(h) degenerate nucleotide sequences corresponding to (a), (b), (c), (d), (e), (f or (g) above.

Further features of the nucleic acid sequence may be as described hereinbefore in relation to the polypeptides of the invention the nucleic acid sequence having the appropriate nucleotide sequence which on expression provides a polypeptide which possesses these features.

In eleventh aspect the invention provides a vector comprising a nucleic acid as hereinbefore defined.

In twelfth aspect the invention provides a host cell transformed with a vector or a nucleic acid molecule as hereinbefore defined.

In thirteenth aspect there is provided a method of making a polypeptide of the invention comprising transforming a host cell with a nucleic acid encodin2 said polypeptide, culturing the transformed cell for and expressing said polypeptide. Expression may be direct or as a fusion product. When the polypeptide product is expressed as a fustion then the method preferably includes a step wherein the expressed polypeptide product is subject to cleavage. The polypeptide may be recovered from the transformed cells expressing it.

In fourteenth aspect the invention provides a pharmaceutical formulation comprising a polypeptide of the invention, optionally together with a diluent, carrier or excipient.

The active ingredients of a pharmaceutical composition comprising the polypeptide are contemplated to exhibit excellent therapeutic activity, for example, in the alleviation of Alzheimer's disease when administered in amount which depends on the particular case. Dosage regima may be adjusted to provide the optimum therapeutic response. For example, several divided doses may be administered daily or the dose may be proportionally reduced as indicated by the exigencies of the therapeutic situation.

The active compound may be administered in a convenient manner such as by the oral intravenous (where water soluble), intramuscular, subcutaneous, intranasal, intradermal or suppository routes or implanting (eg using slow release molecules). Depending on the route of administration, the active ingredient may be required to be coated in a material to protect said ingredients from the action of enzymes, acids and other natural conditions which may inactivate said ingredient.

In order to administer the polypeptide by other than parenteral administration, it will be coated by, or administered with, a material to prevent its inactivation. For example, the polypeptide may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active compound may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof. and vegetable oils. The proper fluidity can be maintained. for example, by the use of a coating such as lecithin. by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required. followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the polypeptide is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipients and used in the form of ingestible tablets, buccal tablets, troches, capsules. elixirs. suspensions, syrups. wafers. and the like. The amount of active compound in such therapeutically useful compositions in such that a suitable dosage will be obtained.

The tablets, troches, pills, capsules and the like may also contain the following: a binder such as gum tragacanth. acacia, corn starch or gelatin; excipients such as dicalcium phosphate; a disintegrating agent such as corn starch, potato starch, aleinic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredient is compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In ninth aspect there is provided polypeptide of the invention as hereinbefore defined for use in the treatment of disease. Consequently there is provided the use of a polypeptide of the invention for the manufacture of a medicament for the treatment of disease associated with aberrant protein/polypeptide structure. The aberrant nature of the protein/polypeptide may be due to misfolding or unfolding which in turn may be due to an anomalous eg mutated amino acid sequence. The protein/polypeptide may be destablished or deposited as plaques eg as in Alzheimer's disease. The disease might be caused by a prion. A polypeptide-based medicament of the invention would act to renature or resolubilise aberrant, defetive or deposited proteins.

In fifteenth aspect there is provided a nucleic acid molecule in accordance with other aspects of the invention for use in the treatment of disease. Consequently, there is provided the use of a nucleic acid molecule of the invention for the manufacture of a medicament for the treatment of disease associated with protein/polypeptide structure. Genetic therapy in vivo is therefore provided for by way of introduction and expression of DNA encoding the monomeric polypeptide in cells/tissues of an individual to provide chaperonin activity in those cells/tissues.

In sixteenth aspect there is provided the use of a polypeptide of the invention for altering the structure of a molecule, particularly a protein or polypeptide and the alteration in structure may be by folding, unfolding or resolubilsing. Preferably, the stoichiometry between the monomeric polypeptide of the invention and the molecule being altered is about 1:1, preferably 1:1.

In seventeenth aspect there is provided a method of reconditioning a molecule preferably a protein comprising contacting said protein with a polypeptide of the invention. Preferably the protein is subjected to inactivation or denaturation prior to contacting with said polypeptide. The polypeptide may be irmmobilised to a solid phase, preferably a chromatographic matrix, and the contacting of protein and polypeptide is carried out by applying the protein to the top of a bed of the matrix packed in a column and then eluting the polypeptide through the column.

This aspect of the invention therefore provides for the use of a polypeptide as hereinbefore defined for altering the structure of a molecule, preferably a protein or polypeptide and the alteration in structure is by folding, unfolding or refolding. The stoichiometry between the polypeptide and the molecule being altered may be about 1: 1.

In nineteenth aspect the -invention provides for the use of a polypeptide of the invention the purification or increase in yield, specific activity or quality of biological molecules, preferably said polypeptide being attached to a support.

In twentieth aspect the invention provides a kit for reconditioning or refolding a molecule, preferably a protein, comprising a polypeptide as hereinbefore defined immobilised to a solid phase and a container for holding said solid phase polypeptide.

In twenty-first aspect the invention provides for the use of a polypeptide as hereinbefore defined in the production of a protein or polypeptide by recombinant means, wherein the said polypeptide is co-expressed with the protein or polypeptide thereby to improve the yield or quality of the protein or polypeptide.

In twenty-second aspect the invention provides an antibody reactive against a polypeptide of the invention. The invention also provides for the use of the antibody in the treatment of disease. Also, the invention provides for the use of the antibody in the manufacture of a medicament for the treatment of disease associated with protein/polypeptide structure.

In twenty-third aspect the invention provides various methods of treating disease, namely a method of treating disease in which an effective amount of a polypeptide of the invention is administered. There is also a method of treating disease which comprises administering an effective amount of an inhibitor of the chaperone activity of a polypeptide of the invention. Preferably, said inhibitor is an antibody. Also, there is a method of treating disease by gene therapy which utilises a construct encoding a polypeptide of the invention or an antagonist thereof.

The momomeric apical domain of GroEL or its fragments catalyse amide proton exchange of barnase, and facilitate refolding of rhodanese and cyclophilin A in the absence of cofactors. Thus, GroEL has an intrinsic chaperone activity, which is not restricted to its oliqomeric state or central cavity. The C-termninal α-helices of the apical domain are not directly involved in polypeptide binding. and form a separate folding unit. At physiological temperature, about 50% of the C-terminus is unfolded, allowing high mobility movements of the polypeptide binding “domain core”. Flexibility within the apical domain may be crucial for cooperative binding of a wide range of proteins with different native structure, and for the conformation change of GroEL on binding to the co-chaperonin GroES. The crystal structure of the “domain core” of the apical domain has an overall B-factor 60% lower than that of the same region in intact GroEL. The overall fold of the fragment is similar to the corresponding region of intact GroEL, but the amount of secondary structure is considerably larger. There are three 3₁₀-helices in the structure of the fragment, which are involved in binding of polypeptide and/or GroES.

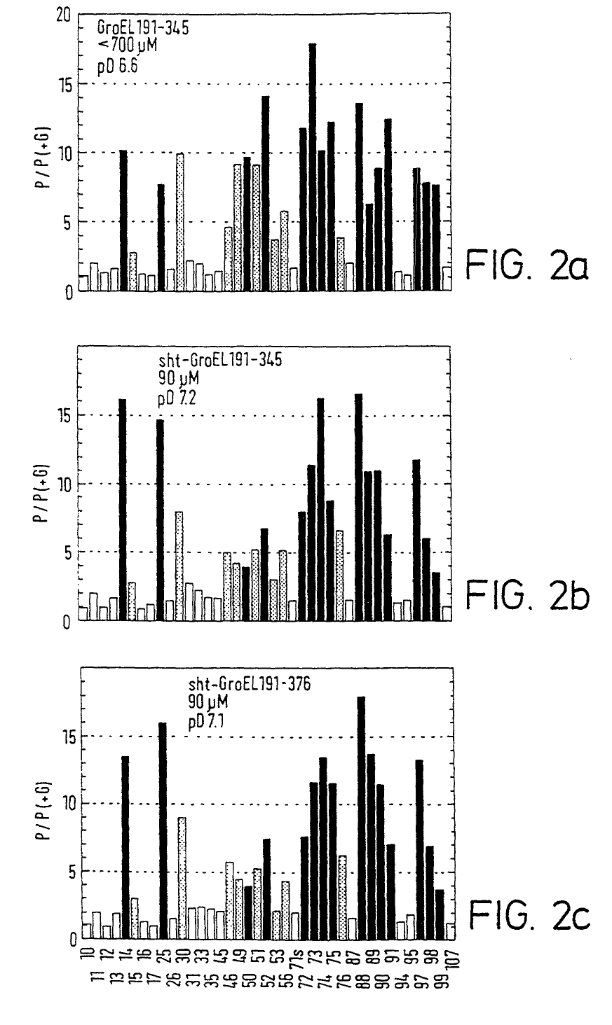

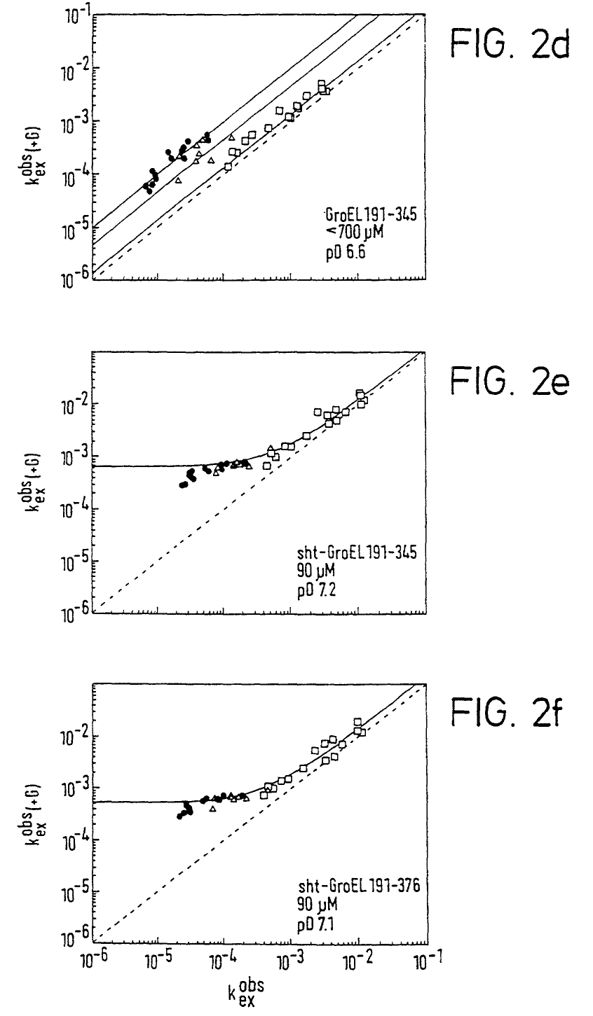

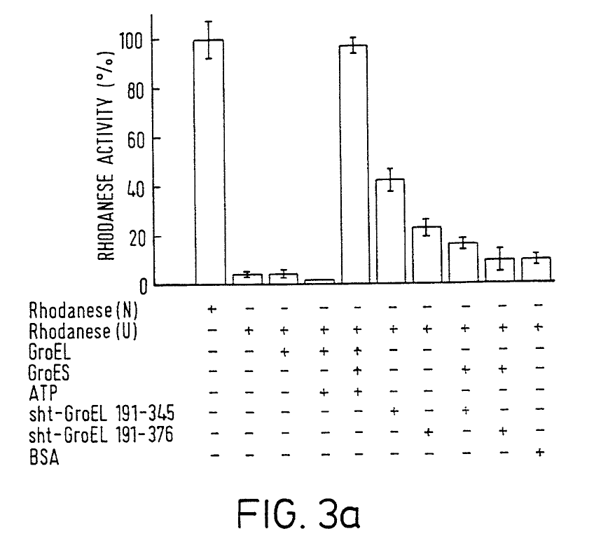

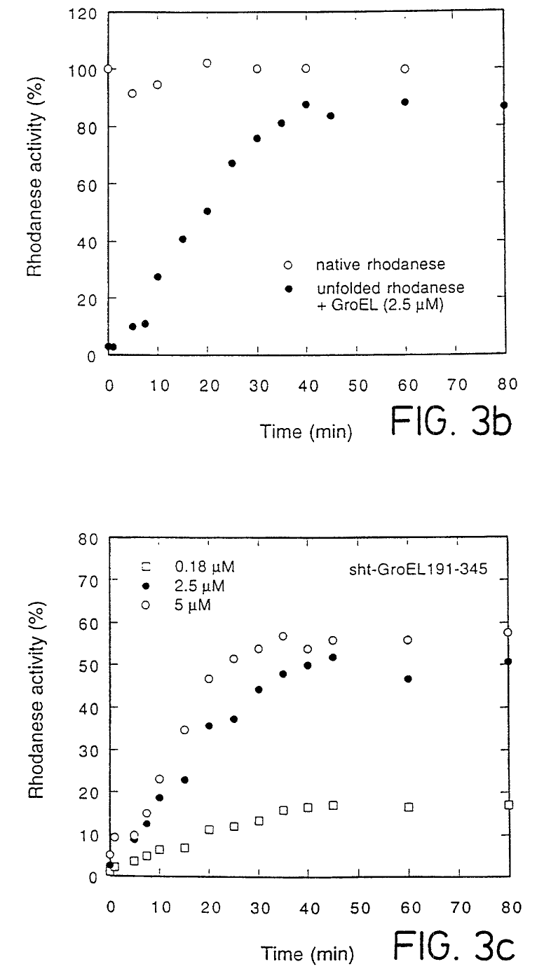

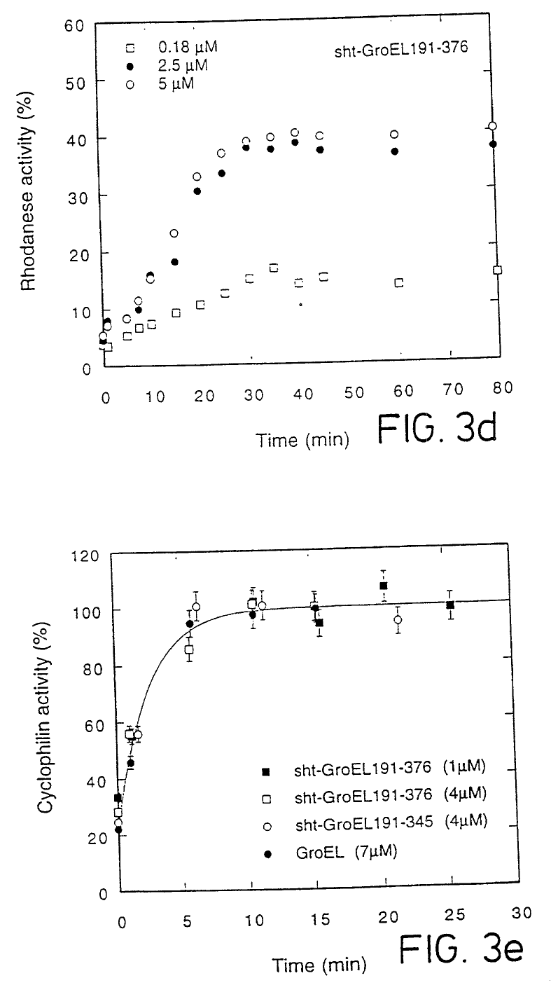

The inventors have expressed the apical domain of GroEL as a stable monomeric protein and with high yield in E. coli, allowing its activity, folding, and structure to be studied separately from those of the equatorial and intermediate domains. The isolated. apical domain is functional in polypeptide binding (FIG. 2), and it facilitates protein folding even when truncated to remove its C-terminal α-helices, H11 and H12 (FIG. 3). The apical domain slows down spontaneous refolding of rhodanese and cyclophilin A by >15-fold and >150-fold, respectively (FIGS. 3c-e). The inventors have shown increased refolding yield of rhodanese and cyclophilin A in the presence of apical domain (FIG. 3), which is too small to reassociate and to form a cavity, clearly demonstrates the presence of an intrinsic chaperone activity in GroEL, which is independent of a central cavity. This is in agreement with NMR experiments on barnase in the presence of apical domain (FIG. 2). The presence of micromolar concentrations of GroEL considerably broadens the resonance lines of barnase owing to a fast association and idssociation of native barnase from the slowly rotational tumbing GroEL (Zahn et at (1996) Science 271: 642-645). If the apical domain and barnase would form a complex of large molecular size, i.e. containing seven or 14 molecules of apical domain, one would expect a considerable degree of line broadening in the NMR spectra of barnase in the presence of apical domain. However, this is not what was observed, even not at eight-fold higher monomer concentration of apical domain than of GroEL (Zahn et at (1996) Science 271: 642-645). The complex between apical domain and barnase, therefore, rather seems to be of low molecular weight and probably has a 1:1 stoichiometry, which would be consistent with the binding stoichiometry of the apical domain and cyclophilin A (FIG. 3e).

The intrinsic chaperone activity of monomeric apical domain facilitates refolding of rhodanese even though physiological conditions, GroES, and ATP are required in the presence of intact GroEL. Although the inventors do not wish to be bound by any particular hypothesis the role of GroES appears to be to weaken the affinity of GroEL for substrates, and to prevent premature dissociation of aggregation prone states: there has to be a balance between tight binding for the annealing activity and weaker binding to allow folding (Corrales F J and Fersht A R (1996) Proc Natl Acad Sci USA 93-. 4509-4512). The weaker binding of rhodanese to the fragment must be adequate for chaperoning activity and weak enough for folding. This is in agreement with the observed similar rate constant for refolding of rhodanese in the presence of apical domain and in the presence of GroEL, GroES, and ATP (FIGS. 3b-d), indicating a similar binding affinity. The complex structure of GroEL, and the modulation of its substrate affinity by GorES and nucleotides, must be to allow the efficient folding of a wide range of proteins, with a wide variation of affinities for GroEL. The mechanism. in general, may involve several components: GroES binds to the cis-end of a GroEL-substrate complex and displaces the bound polypeptide into the cavity; the dissociated and encapsulated polypeptide chain then refolds in an enlarged folding cavity to a native-like state, until it is released upon the dissociation of GroES; and, the cycle of binding and dissociation of unfolded protein, GroES, ATP, and GroEL may be repeated several times, before the substrate protein can adopt a native conformation (Hunt et al (1996) Nature 37: 37-45; Weissman et al (1996) Cell 84: 481-490; Weissman J S et a! (1995) Cell B: 577-587: Mayhew M et al (1996) Nature 379: 420-426; Corrales F J and Fersht A R (1996) Proc Natl Acad Sci USA 9: 4509-4512). Polypeptides with intermediate affinity for GroEL, such as cyclophilin A (FIG. 3e), bind transiently to GroEL and folding is facilitated without the need for co-factors.

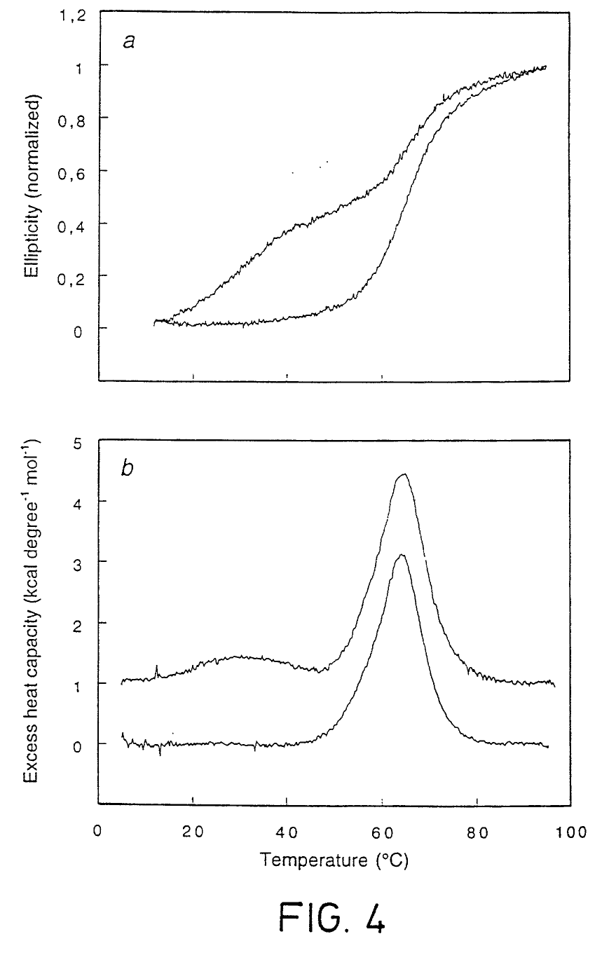

Helices H11 and H12 are much less stable than the “domain core” of the apical domain, and form a separate folding unit (FIG. 4). At physiological temperature, about 50% of the secondary structure of the low-melting helices is unfolded and thus becomes flexible. But, in vivo, the C-terminus is not degraded, implying that the primary structure is not accessible to degradation by E. coli proteases. The low stability of the C-terminal helices allows movements and larger fluctuations of the “domain cores” within the GroEL-ring. This flexible arrangement of the apical domain may be implicated in the cooperative binding of unfolded polypeptide to the seven subunits of GroEL in each ring. It is understandable that flexibility is required for binding to a large variety of proteins with different amino acid sequence, and secondary and tertiary structure. The observed low stability of the C-terminus may also contribute to the large conformational chance in GroEL on binding of GroES, which has been suggested to occur through rigid body movement through the β-sheet hinge region of the intermediate domain (Braig K et al (1994) Nature 371: 578-586).

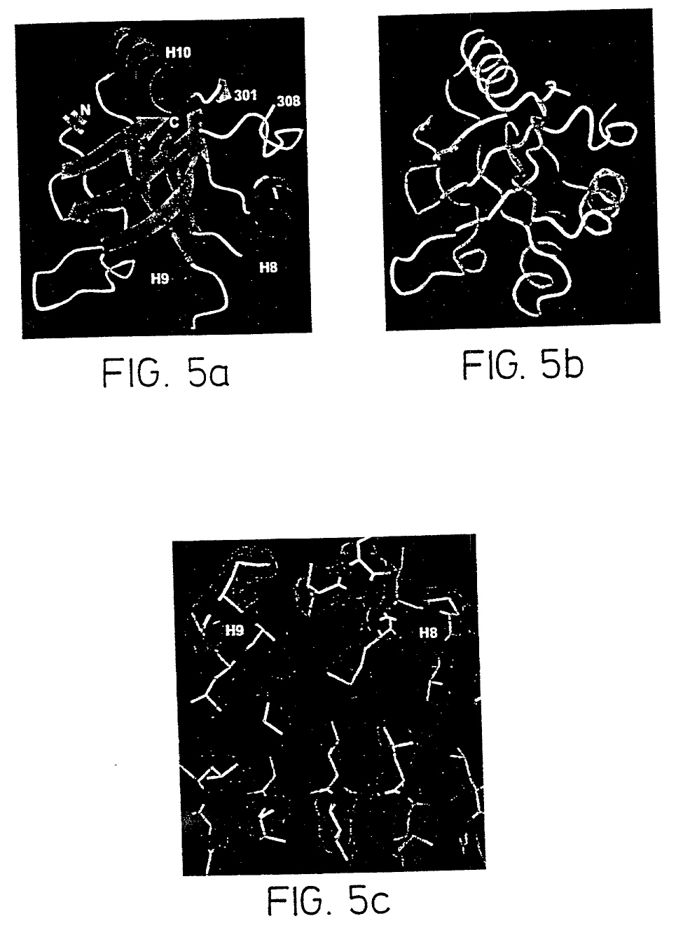

The production and crystallisation of monomeric fragments of the apical domain by the inventors has allowed a determination of the three-dimensional structure of the polypeptide binding part of GroEL with B-factors much lower than those of the equivalent regions in GroEL (FIG. 5) (Braig K et al (1994) Nature 371: 578-586; Braig et al (1995) Nature Struct Biol 2: 1083-1094; Boisvert et af (1996) Nature Structure Biology 3: 170-177), perhaps because of favourable packing interactions within the crystal, or the absence of the flexible C-termini in the GroEL fragment, or a combination of both. Particularly interesting is the existence of 3₁₀-helices within the apical domain. These tightly packed helices are much less common than α-helices. and the majority are very short, with 96% being less than four residues. They are usually found at the end of α-helices. where the final turn may have this conformation, or between two β-strands. All the 3₁₀-helices of the apical domain are localised within regions, which have been shown to be involved in polypeptide and/or GroES binding (Fenton et al (1994) Nature D: 614-619). The third 3₁₀-helix has an unusual length of 10 residues. A 3₁₀-helix with a similar length (9 residues) has been found in aconitase (Robbins et at (1989) Proteins: Structure, Function and Genetics 5: 289-312), an enzyme of the Calvin cycle. Interestingly, the apical domain and aconitase also share a similar fold: a central β-sheet linked by α-helices, suggesting that they may use a common mechanism for substrate binding.

Preferred embodiments of the invention will now be described in detail by way of the following examples and with reference to the accompanying drawings in which:

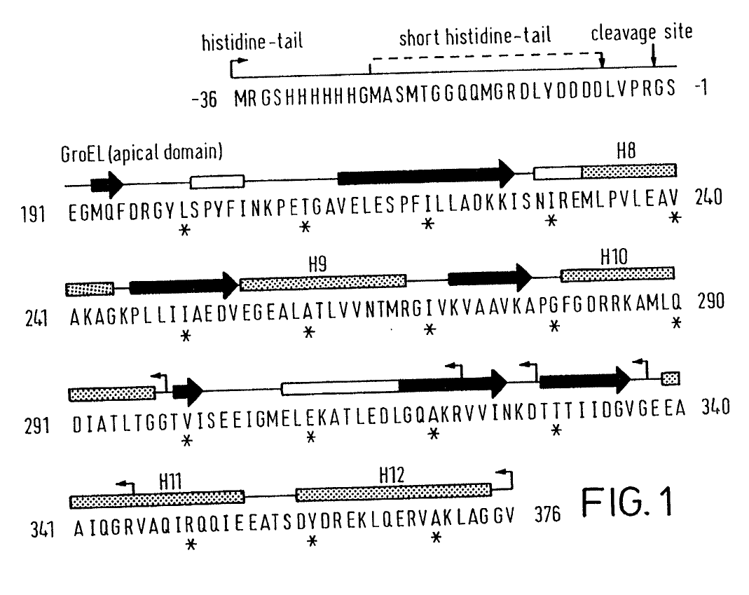

FIG. 1 shows the amino acid sequence of the apical region of GroEL from E. coli (SEQ ID NO:9) including further amino acid residues providing an N-terminal histidine-tail (ht)(SEQ ID NO:8).

FIG. 2a is a bar chart showing the catalytic effect of GroEL 191-345 on the amide proton exchange of unfolded barnase. The ratio of the protection factors of the 39 measurable amide protons of barnase in the absence (P) and presence (P+(G)) of the GroEL fragment is shown.

FIG. 2b is a bar chart similar to that of FIG. 2a but for the GroEL fragment 191-345 with a 17 residue N-terminal histidine tail (sht-GroEL 191-345).

FIG. 2c is a bar chart similar to that of FIG. 2a but for the GroEL fragment 191-376 with a 17 residue N-terminal histidine tail (sht-GroEL 191-376).

FIG. 2d is a plot of observed exchange rate constants in the absence of chaperonin (k^{obs}ₑₓ) against those in the presence of chaperonin (k^{obs}ₑₓ(+G)) for GroEL fragment 191-345. Global, mixed and local amide protons are displayed by circles, triangles and squares respectively.

FIG. 2e is a plot similar to that of FIG. 2d but for sht-GroEL 191-345.

FIG. 2f is a plot similar to that of FIG. 2d but for sht-GroEL 191-376.

FIG. 3a is a bar chart and table showing the relative enzymatic activity of rhodanese after refolding in the presence or absence of GroEL, GroES, ATP, sht-GroEL 191-345, sht-GroEL 191-376 or bovine serum albumin (BSA).

FIG. 3b is a plot showing the refolding kinetics of rhodanese in the presence of GroEL, GroES and ATP.

FIG. 3c is a plot showing the refolding kinetics of rhodanese in the presence of various concentrations of sht-GroEL 191-345.

FIG. 3d is a plot showing the refolding kinetics of rhodanese in the presence of various concentrations of sht-GroEL 191-376.

FIG. 3e is a plot comparing the refolding kinetics of specified concentrations of sht-GroEL 191-376, sht-GroEL 191-345 and GroEL.

FIG. 4a is a trace of the thermal denaturation of sht-GroEL 191-376 (upper trace) and sht-GroEL 191-345 (lower trace) monitored by far ultra violet-circular dichroism at 222 nm.

FIG. 4b is a trace as in FIG. 4a but thermal denaturation is monitored by differential scanning calorimetry.

FIG. 5a shows a three dimensional representation of the structure of sht-GroEL 191-345.

FIG. 5b shows a three dimensional representation of the backbone structure of sht-GroEL 191-345.

FIG. 5c shows a three dimensional representation of election densities of sht-GroEL 191-345 viewed along the helices H8 and H9.

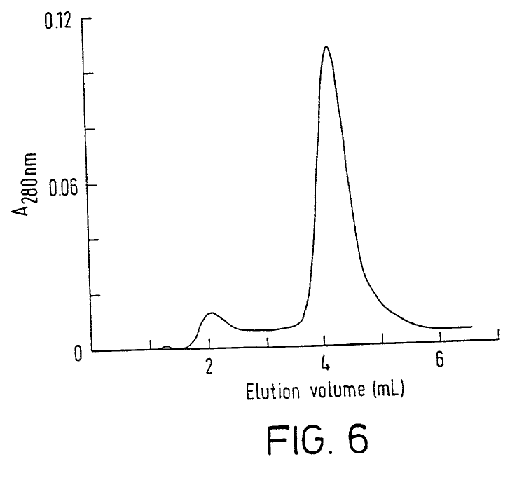

FIG. 6 is a trace showing the elution of denatured mutant IGPS from a column of inmmobilised GroEL 191-345 developed with refolding buffer.

FIG. 7 shows the amino acid sequence of GroEL from E. coli numbered from the N-terminal methionine (SEQ ID NO: 10).

FIG. 8 shows the amino acid sequence of a portion of GroEL wherein the residues involved in binding peptides are highlighted (SEQ ID NO:1l).

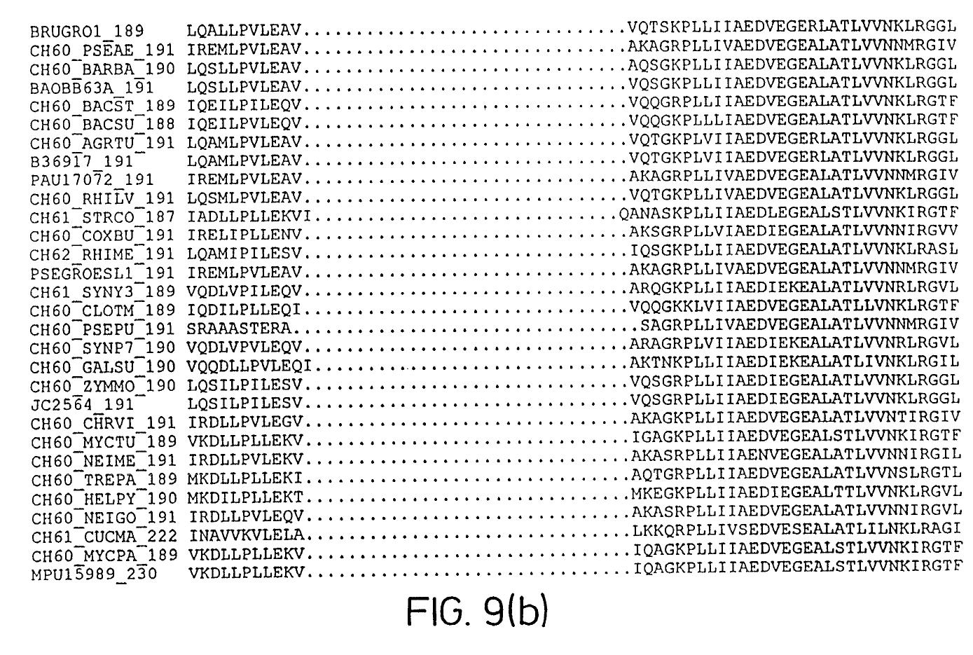

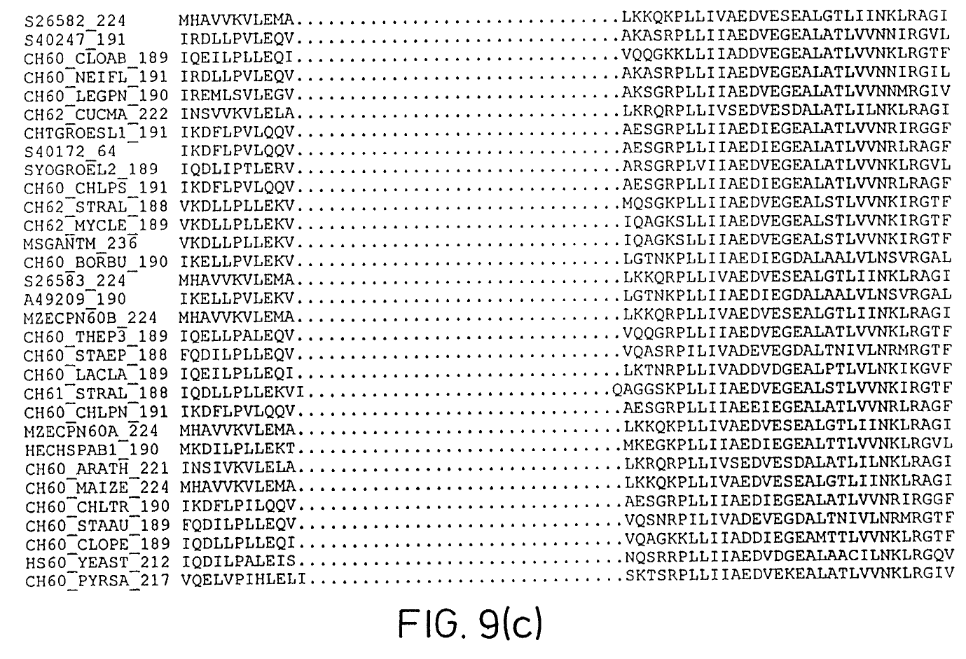

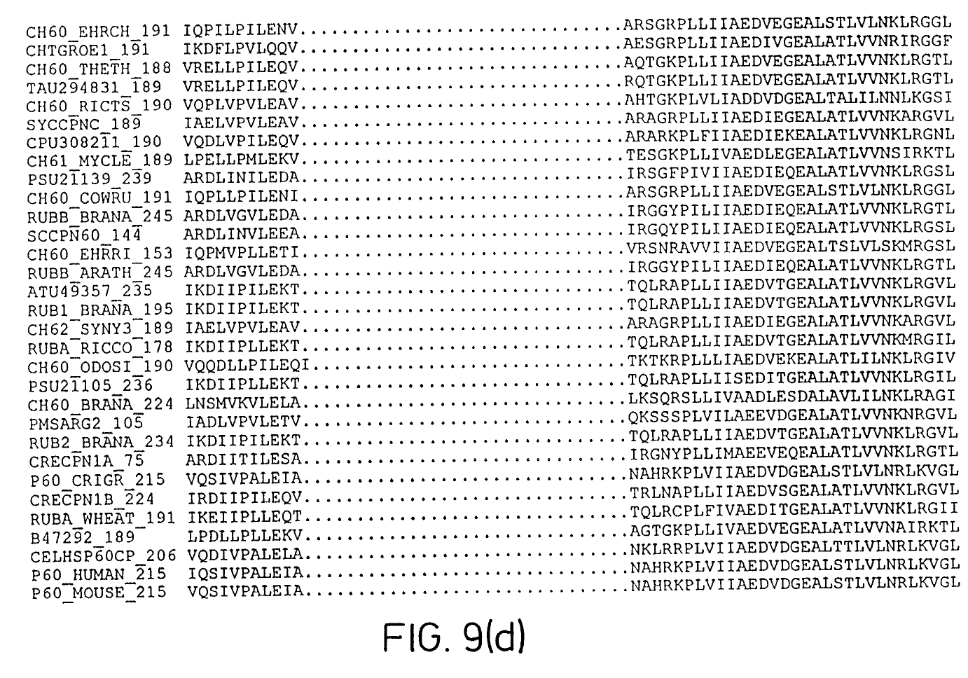

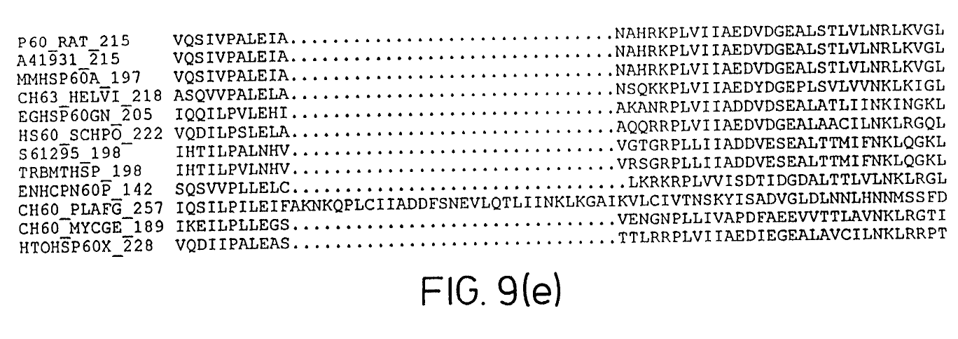

FIGS. 9a-c comprise partial amino acid sequences of known cpn 60 family members aligned with one another for comparison.

EXAMPLE 1

Cloning and expression of the apical domain of GroEL and various fragments thereof

The apical domain of GroEL (GroEL 191-376) and various C-terminally truncated fragments of the apical domain are cloned by polymerase chain reaction (PCR) into the polylinker site of a pRSET A vector (Invitrogen), coding for an N-terminal histidine-tail, which contains an engineered thrombin cleavage site. The histidine-tail is composed of 36 amino acids (Invitrogen) or 17 amino acids. For the PCR reaction, the plasmid pOF39 (Fayet 0 et al (1989) J Bacteriol 171: 1379-1385) is used as a template together with two primers flanking the respective GroEL fragment with BamHI and CeoRI restriction enzyme sites. This permits the cloning of the PCR fragment into the polylinker of pRSET A. PCR is performed with PSfu (Stratagene) to reduce the risk of undesired random mutations. The reaction is performed in a volume of 25 μl for 25 cycles with 400 nM or primer and 200 μM of deoxynucleoside-5′-triphosphates. The annealing temperature is 65° C. The following primers are used for the PCT to generate DNA encoding the apical region of GroEL and a variety of fragments thereof: 5′ flanking: 5′-CGG ATC CGA AGG TAT GCA GTT CGA CCG (SEQ ID NO:1); 3′ flanking (s)ht-GroEL191-376, 5′-CGA ATT CTT AAA CGC CGC CTG CCA GTT TCG (SEQ ID NO:2); 3′ flanking (s)ht-GroEL191-345, 5′-CGA ATT CTT AAC GGC CCT GGA TTG CAG CTT C (SEQ ID NO:3); 3′ flanking ht-GroEL191-337, 5′-CGA ATT CTT AAC CCA CGC CAT CGA TGA TAG TGG TG (SEQ ID NO:4); 3′ flanking ht-GroEL191-328. 5′-CGA ATT CTT AGT CTT TGT TGA TCA CAA CAC GTT TAG CCT GAC (SEQ ID NO:5); 3′ flanking ht-GroEL191-322. 5′-CGA ATT CTT AAC GTT TAG CCT GAC CCA GGT CTT CCA (SEQ ID NO: 6); 3′ flanking ht-GroEL191-298, 5′-CGA ATT CTT AAC CGO CAG TCA GGG TTG CGA TAT C (SEQ ID NO: 7).

The following protocol is used for expression and purification of the fragments of GroEL in E. coli TG2 cells. With reference to FIG. 1. the expression vector used codes for an N-terminal histidine-tail (ht) composed of 36 amino acids and containing a thrombin cleavage site (vertical arrow). Alternatively, a shorter version of this histidine-tail (sht) containing 17 amino acids is used. The N- and C-terminal ends of the generate fusion proteins, namely ht-GroEL 191-298, ht-GroEL 191-322, ht-GroEL 191-328, ht-GroEL 191-337, ht-GroEL 191-345, ht-GroEL 191-376, sht-GroEL 191-345, and sht-GroEL 191-376 are indicated by rectangular arrows.

Two litres of L-Broth medium plus ampicillin is inoculated 1:100 with an over-night culture of TG2 cells containing the respective plasmid. At an A₆₀₀ of 0.3, expression is induced with isopropyl-1-thio-β-D-galactopyranoside to 0.2 mM final concentration and M13/T7-phage at a multiplicity of infection of 10 phages per cell. The cells are harvested 8 h after induction, centrifuged, and resuspended in 200 ml buffer A (50 mM Tris-HCl pH 8.2. 300 mM NaCl).

After sonication and centrifugation. the soluble protein fraction is added to 20 ml nickel-NTA agarose resin and stirred for 10 min. The resin is washed with 200 ml buffer A, and the histidine-tail containing fusion protein is eluted with 50 ml buffer

A containing 200 mM imidazole. The eluted protein is precipitated with 80% ammonium sulfate, re-dissolved in 4 ml of buffer B (50 mM Tris-HCl pH 8.2, 150 mM sodium chloride), and is loaded on a HiLoad 26/60 Superdex 75 column (Pharmacia), which is equilibrated with buffer B. The fragment GroEL 191-345 is produced by thrombin-cleavage of ht-GroEL 191-376 before gel filtration. The cleaving reaction is carried out for several days in buffer B after addition of 250 μl thrombin (Sigma, 1U/μl) to the protein solution eluted from the nickel-NTA column. The GroEL fragments are analyzed by quantitative amino acid analysis, N-terminal sequencing, and mass spectrometry.

In FIG. 1, secondary structure is indicated by boxes and arrows for α-helix (grey) or 3₁₀-helix (white) and β-sheet structure. respectively. Assignment of secondary structure of residues 191 to 336 is done on the basis of the crystal structure of sht-GroEL 191-345 (Table 1 and FIG. 5) using PROCHECK (Laskowski R A et al (1993) J Appl Cryst 2&: 283-291) and the algorithm of Kabsch & Cander (Kabsch W and Sander C (1983) Biopolymers L: 2577-2637). Numbering of α-helices and secondary assignment of residues 337 to 376 according to Braig et al (Braig K et al (1995) Nature Struct Biol 2: 1083-1094).

Functional apical domain of GroEL and various functional fragments of the apical domain in E. coli are expressed allowing the study of chaperone activity, folding, and crystal structure of the polypeptide binding part of GroEL. In particular, the apical domain of GroEL (GroEL 191-376) and various fragments of the apical domain (FIG. 1) are expressed in E. coli as fusion proteins containing an N-terminal histidine-tail. and this allowed for a straightforward purification using a nickel-nitrilo-tri-acetic acid (NTA)-ligated agarose resin. The histidine-tail of either 39 (ht) or 17 amino acids (sht) contains a sequence of six histidine residues and a thrombin cleavage site. The apical domain is expressed at >20 mg purified protein per litre of culture as are the smaller fragments lacking the C-terminal α-helices, H11 and H12. Further truncation before residue 329 leads to considerable destabilisation of the apical domain. The fragment GroEL 191-345. containing no histidine-tail and without the two helices is generated by thrombin cleavage of purified ht-GroEL 191-376.

The apical region and monomeric fragments are found to have chaperone activity. The circular dichroism (CD) of sht-GroEL 191-376, sht-GroEL 191-345, and GroEL 191-345 in the far ultra violet (UV) region and in the near UV region indicated native-like secondary and tertiary structure, respectively. The apical domain and the fragment truncated at position 345 are monomeric at micromolar concentrations, when determined by ultracentrifugation. However. the line widths in the nuclear magnetic resonance (NMR) spectra of GroEL 191-345 are larger than expected for a 17 kD protein but small than for a stable dimer (data not shown), indicating a fast intermolecular interaction between monomers on the NMR time scale (Wuithrich, K, NMR of Proteins and Nucleic Acids (Wiley, New York, 1986). There seems to be a low affinity self-recoanition of the apical domain.

### EXAMPLE 2

Bindino of GroEL 191-345 . sht-GroEL 191-345. and sht-GroEL 191-376 to unfolded barnase

Exchange experiments are carried out at 33° C. in 20 mM imidazole buffer as described in Zahn et al (1996) Science 2l: 642-645. The concentration of barnase is 2.4 mM. The results are shown in FIGS. 2a, 2b and 2c; locally and globally exchanging amide protons, and amide protons exchanging by a mixture of both mechanisms (Perrett et al (1995) Biochemistry 34: 9288-9298 and Clarke et al (1993) Proc Natl Acad Sci USA 2Q: 9837-9g41) are indicated by white, black, and grey bars, respectively. The indole NH proton of Trp71 is indicated by 71s.

The same experiments are shown in FIGS. 2d, 2e and 2f respectively. The data in FIG. 2d is fitted to k^{obs}ₑₓ(+G)=Ck^{obs}ₑₓ, where C is a factor by which EX2 mechanism exchange is catalysed by GroEL fragment. The values of C for the globally, locally, and mixed amide protons of barnase are 10, 5, and 1. The data in FIGS. 2e and 2f is fitted to k^{obs}ₑₓ(+G)=Ck^{obs}ₑₓ+k^{obs}ₑₓ(G.U), where k^{obs}ₑₓ(G.U) is the observed EX1 rate constant for global exchange of barnase in the presence of GroEL fragment. FIGS. 2d, 2e and 2f serve to demonstrate a mechanism of amide proton exchange of barnase. In FIG. 2a and 2d there is only an estimate of the final concentration of GroEL fragment. At the high initial protein concentration used, GroEL 191-345 tended to crystallise during the exchange experiment.

FIGS. 2a , 2b and 2c show that the GroEL fragments catalyses under folding conditions, exchange of amide protons of native barnase that are known to exchange only from its fully unfolded state (Perrett et al (1995) Biochemistry 34: 9288-9298). Thus. like intact GroEL (Zahn et al (1996) Science 271: 642-645), the apical domain binds with high affinity to unfolded barnase, and helices H11 and H12 are not essential for polypeptide binding (FIGS. 2a and 2b). The presence of the N-terminal histidine-tail does not abolish binding activity (FIGS. 2b and 2c).

The mechanism of exchange at pD 6.6 is EX2 (FIG. 2d), but changes to EXI at higher pD (FIGS. 2e and 2f). In an EX2 mechanism (Hvidt A and Nielsen S O (1966) Advan Protein Chem 21: 287-386), the observed rate constant is limited by the intrinsic rate constant for exchange, which depends on pD. The criterion for an EX2 mechanism is that the rate constant for reprotection (ie a refolding or dissociation reaction) is much greater than the intrinsic rate constant. At the other extreme, when intrinsic exchange is much faster than reprotection the mechanism becomes EX1 (Hvidt A and Nielsen S O (1966) supra) and the observed rate constant is simply equal to the rate constant for the formation of the unprotected state. Thus from the exchange behaviour (and assuming that the presence of the histidine-tail does not effect binding) the rate constant for dissociation of barnase from the apical domain is about 2 s⁻¹, which is less than five-fold larger than that from intact GroEL (Zahn et al (1996) supra).

### EXAMPLE 3

Refoldino of rhodanese

Rhodanese refolding assays are performed using GroEL, GroES, ATP, sht-GroEL 191-345 and sht-GroEL 191-376 and carried out as described by Horowitz (Horwitz P M in Protein Stability and Folding (eds Shirley B A) 361-368 (Humana Press, 1995)).

In more detail. rhodanese (9 μM) is unfolded for 45 min at 25° C. in the presence of 8 M urea and 1 mM β-mercaptoethanol. Refoldina is initiated by diluting 3 μl of the unfolded rhodanese into a final volume of 250 μl of a standard buffer containing 50 mM Tris-HCl pH 7.8, 50 mM sodium thiosulphate. 10 mM MgCl₂, 10 mM KC1. GroEL. GroES. ATP, apical domain (or its C-truncated form) and bovine serum albumin (BSA) are included as indicated, at final concentrations of 2.5 μM monomer, 2.5 μM monomer, 2 mM, 2.5 μM and 45 μg/ml (the same concentration by weights as the GroEL fragments), respectively.

After incubation for 50 min at 25° C., rhodanese activity is measured by adding 25 A¹ from the refolding mixture to 1 ml of 50 niM Na₂S₂O₃ 50 mM KCN and 40 mM potassium phosphate buffer pH 8.6. The reaction is terminated after 15 min incubation by addition of 0.5 ml of 18% formaldehyde. Colour is developed by mixing with 1.5 ml of ferric nitrate reagent (400 g FeNO₃.9H₂); 800 ml 65% HNO₃ in a final volume of 3 dm³) prepared as indicated (Perrett et al (1995) supra).

FIG. 3a shows the relative enzymatic activity of rhodanese (0.1 μM) after refoldin2 in the presence (+) or absence (−) of GroEL (2.5 μM monomer), GroES (2.5 AM monomer), ATP (2 mM), sht-GroEL 191-345 (2.5 μM), sht-GroEL 191-376 (2.5 μM), or bovine serum albumin (BSA; 45 μg/ml), from 8 M urea (U). 100% activity is obtained with native rhodanese (N) alone.

FIG. 3b shows the refolding kinetics of rhodanese in presence GroEL, GroES, and ATP (•). The final concentrations are the same as in FIG. 2a. 100% activity is obtained with native rhodanese (O).

FIGS. 3c and 3d show the refolding kinetics of rhodanese in the presence of 0.18 μM ( ), 2.5 μM (•), or 5 μM (O) sht-GroEL 191-345 and sht-GroEL 191-376, respectively.

FIG. 3a shows a rhodanese refolding activity of about 42.5% for shtGroEL 191-345 which is about 37.5% above background refolding as shown by the control of unfolded rhodanese (U) alone.

The results of FIG. 3b are for comparitive purposes and show a time course of refolding activitv for GroEL, GroES and ATP.

FIG. 3c shows the rhodanese refolding kinetics at various concentrations of sht-GroEL 191-345. Refolding activities of about 50% are achieved at about 25 mins.

FIG. 3d is similar to FIG. 3c but for sht-GroEL 191-376 and showing refolding activites of about 40% after 25 mins.

Enzymatic activity is obtained from the absorbance at 460 nm of the complex formed between thiocyanate and ferric ion. Results correspond to the average of three different independent assays. Standard error bars are shown. b-d, same as in a, but, to stop the refolding reaction rhodanese activity is assayed in the presence of 10 mM trans-1,2-cyclohexanediaminetetraacetate (CDTA) to inhibit GroEL activity or 0.5 mg/ml of casein to saturate the apical domain.

Maximal refolding yield is obtained at molar ratios of apical domain and rhodanese of larger than one, from which is estimated a dissociation equilibrium constant of >1×10⁻⁷ M.

### EXAMPLE 4

Refolding of cvclophilin A

The chaperone activity of sht-GroEL 191-345 and sht-GroEL 191-376 is tested using cyclophilin A. Refolding of cyclophilin A is initiated by diluting 8 M urea denatured protein (100 μM) into 100 mM potassium phosphate buffer pH 7.0, 10 mM DTT to a final concentration of 1 μM. The final concentration of GroEL and apical domain in refolding buffer is 7 μM and 4 μM or 1 μM, respectively. Refolding temperature is 25° C. After incubation for the times indicated, cyclophilin activity is measured as described (Fischer G et al (1984) Biomed Biochim Acta 43: 1101-1111). Spontaneous refolding of cyclophilin A occurred to a yield of about 30%, and is finished in less than 1 min. Standard error is 5%.

FIG. 3e shows the refolding of 1 μM cyclophilin A in the presence of 7 μM GroEL monomer (O,), 4 μM sht-GroEL 191-345 (•), 4 μM sht-GroEFL 191-376 ( ), or 1 μM sht-GroEL 191-376 ( ). 100% activity is obtained with native cyclophilin A. FIG. 3e demonstrates that 100% refolding of inactivated cyclophilin A can be achieved with sht-GroEL 191-345 or sht-GroE 191-376 and that this is equivalent to that seen with GroEL.

Cyclophilin A refolds only at low yield in the absence of chaperone but refolding is facilitated in the presence of GroEL monomer owing to a transient complex formation (see Zahn et al (1996) FEBS Lett 380: 152-156). A similar rate constant is found for refolding of cyclophilin A in the presence of intact GroEL monomer and in the presence of GroEL fragments, which within a factor of four does not depend on chaperone concentration (FIG. 3e). Maximal refolding yield is obtained at stoichiometric concentrations of cyclophilin A and apical domain, indicating the formation of 1:1 complex between chaperone fragment and substrate protein during refolding.

### EXAMPLE 5

Thermal denaturation of sht-GroEL 191-376 and sht-GroEL 191-345

Thermal denaturation monitored by far ultra violet-circular dichroism (UV-CD) at 222 nm and is carried out on a Jasco J720 spectropolarimeter interfaced with a Neslab RTE-100 water bath, using a thermostated cuvette (Helma) with 1 mm path length. The temperature is increased at a linear rate of 50 deglh. The protein concentration is 40 μM in 10 mM sodium phosphate buffer pH 7.0. Data are fitted to a denaturation curve (Pace, C N (1990) Trends Biotechnol 8: 93-98) to determine Tₘ, the midpoint temperature of denaturation.

Thermal denaturation is also monitored by differential scanning calorimetry (DSC) and the measurements are performed at a protein concentration of 88±5 μM in 10 mM sodium phosphate buffer pH 7.0, using a Microcal MC-2D instrument at a notional scan rate of 60 deg/h. Sample preparation and data analysis are performed as described previously (Johnson C M and Fersht A R (1995) Biochemistry 34: 6795-6804). Both proteins exhibit at least 50% reversibility in their thermal unfolding as judged from the area of endotherms obtained on rescanning samples. Higher levels of reversibility are obtained at lower concentrations or by stopping scans at temperatures closer to the main unfolding transition. The low temperature transition observed in sht-GroEL 191-376 is completely reversible with scans limited in temperatures below 50° C.

FIG. 4a shows the far UV-CD results and FIG. 4b shows the DSC results. In each Figure. sht-GroEL 191-376 is the upper trace and sht-GroEL 191-345 is the lower trace.

The C-terminal helices are found to be flexible. The apical domain and the fragment truncated at position 345 are reversibly denatured by temperature or urea, and the denaturation is not influenced by the N-terminal histidine-tail. There are two cooperative folding transitions, at 34° C. and 67° C. At 45° C., the CD spectrum of the apical domain is identical to that of the truncated domain. The C-terminal α-helices, therefore, must melt at the lower temperature and separately from the “domain core”. The second cooperative transition associated with the extra 31 amino acids in sht-GroEL 191-376 is confirmed by DSC (FIG. 4b). The calorimetric data are also essentially consistent with the unfolding of the apical domain as a monomer. At physiological temperature. about 50% of helices Hi1 and H12 are in an unfolded conformation, and thus flexible.

### EXAMPLE 6

Crystallisation of GroEL fragments and diffraction study

Crystals are obtained from hanging drops initially containing sht-GroEL 191-345 at 23 mg ml⁻¹, 11% PEG 4000, 50 mM Tris-HCl pH 8.5 and 100 mM LiSO₄, equilibrated against reservoirs consisting of 22% PEG 4000, 100 mM Tris-HC1 pH 8.5 and 200 mM LiSO₄. X-ray data are collected from a capillary-mounted crystal at 4° C. using a 30 cm Mar Research image plate detector at station 9.6 of the Synchrotron Radiation Source (SRS) at Daresbury, UK (λ=0.87 Å). Unless stated otherwise, all data processing data reduction, electron density syntheses and structural analyses are carried out using CCP4 software (Daresbury Laboratory, Warrington. UK). Indexing and intensity measurements of diffraction data are carried out with the MOSFLM program suite Leslie A G W in Joint CCP4 and ESF-EACMB Newsletter on Protein Crystallography No 26 (Daresbury Laboratory, Warrington, UK, 1992).

The structure is solved by conventional molecular replacement methods, using the program AMORE (Navaza J (1994) Acta Crystallogr A S: 157-163), and a search model consisting of residues 191-345 of the refind structure of GroEL (Braig et al (1995) Nature Struct Biol 2: 1083-1094). The asymmetric unit contains one protein molecule, corresponding to a solvent content of 51%.

Model rebuilding and refinement is carried out with O (Jones et al (1991) Acta 5 Crystallogr A 47: 110-119), and the structure is refined using X-PLOR (Brunger A T X-PLOR, Version 3.1, A System for Crystallography and NMR (Yale Univ Press, New Haven, Conn., 1992)), using Engh and Huber parameters (Engh R A & Huber R (1991) Acta Crystallogr A 47: 392-400). The current model contains 8 water molecules and is complete with the following exceptions, which could not be 10 modelled due to poor or non-existent electron density: residues 302-307 and residues 337-345 from the C-terminus. Electron density for the N-terminal His-tag is also not observed. No residues have disallowed backbone φψ angles. Table 1 provides a sutntnary of the crystallographic data.

TABLE I
Summary of crystallographic data

| Data collection statistics | |
|---|---|
| Unit cell dimensions | a = b '2 91.67 Å, c = 38.33 Å |
| Space group | P3₁21 |
| Resolution (Å) | 22.0-2.5 |
| Measured reflections | 21,762 |
| Unique reflections | 6,564 |
| Completeness of data (%)* | 99.4 (96.7) |
| R_{merge} (%)*^{†} | 9.9 (45.1) |
| <F/σF>* | 19.8 (4.6) |
| Multiplicity* | 3.3 (3.0) |
| Refinement statistics | |
| Resolution (Å) | 8.0-2.5 |
| R-factor/free R-factor (%), F > 0^{‡} | 21.4/29.1 |
| r.m.s.d bond length (Å) | 0.006 |
| r.m.s.d bond angle (deg) | 1.42 |

*values given in parenthesis are for the highest resolution shell.
^{†}agreement between intensities of repeated measurements of the same reflections and can be defined as: Σ(I_{h,i} − <Iₕ>)/Σi_{h,i}, where I_{h,i} are individual values and <Iₕ> is the mean value of the intensity of reflection h
^{‡}The free R-factor is calculated with the 10% data omitted from the refinement (test set, prepared using DATAMAN (Kleywegt, G.J. & Jones, t.A. Acta. crystallogr. D 50, 178-185 (1994)

### EXAMPLE 7

The three dimensional structure of sht-GroEL 191-345

FIG. 5a shows the secondary structure representation drawn with MoIScript (Kraulis P J (1991) J Appl Crystalloor 24: 946-950) and Raster3D (Merrit E A and Murphy M E P (1994) Acta Crystallogr D 50: 869-873). Helices are labelled as in Braig et al (Braig et al (1995) Nature Struct Biol 2: 1083-1094). N and C refer to the N-terminus (residue 191) and C-terminus (residue 336) of the model.

FIG. 5b is a backbone representation drawn with prograrnm O (Jones et al (1991) Acta Crystallogr A 47: 110-119), in same orientations as FIG. 5a colour-coded according to B-factor of main-chain atoms: blue (20 Å²) to red (60 Å²).

FIG. 5c shows a representative region of electron density, calculated using refined co-ordinates, viewed along the helices H8 and H9.

Crystals of sht-GroEL 191-345 grow in the trigonal space group P3₁21 with one molecule per asymmetric unit, giving a solvent content of 51%. The three dimensional structure of the “domain core” is solved by molecular replacement and refined to an R-factor of 21.4% and a free R-factor of 29.1% for all data between 8.0 and 2.5 Å (Table 1). The quality of the electron density map is shown in FIG. 5c.

Overall, sht-GroEL 191-345 has the same fold as the corresponding region of the intact GroEL protein (FIG. 5a): two orthogonal β-sheets forrning a β-sandwich, flanked by three α-helices. The structure is more ordered and better resolved than is the apical domain of the intact protein (FIG. 5b): the average B-factor is 42 Å², compared with 97 Å² for residues 191-336 of the GroEL structure. Unfortunately, such unusually high disorder in the GroEL structure complicates the interpretation of a structural comparison, in the same way as it can be misleading to use an average of NMR structures for comparison with a crystal structure. and so we have not done so here. In essence, the structure can be described as a well ordered β-sandwich scaffold. flanked by relatively flexible helical and loop regions. In particular, the B-factors of most of the β-strand, α-helix, and loop structure is about 20, 40, and 60 Å², respectively (FIG. 5b). There are two regions of considerable disorder. First, electron density is not found for the C-terminal residues 337-345, which correspond to the first half of α-helix H11. This is an agreement with the results from folding experiments, described above. Second, electron density for residues 302-307 is very poor and fragmented. This region is part of the most disordered sesment in the structure of intact GroEL (Braig et al (1995) Nature Struct Biol 2: 1083-1094). The content of α-helix and β-sheet secondary structure of the GroEL fragment (FIG. 1) is 48% and 74% higher. respectively, compared with the corresponding region of intact GroEL. There are four additional segments of secondary structure in the new structure (FIGS. 1 and 5a): residues 299-301 form a short β-strand; residues 201-205, 229-232 and 308-317 form 3₁₀-helices.

### EXAMPLE 8

Refolding chromatography of IGPS (49-252) (indoleglycerol phosphate synthase lacking residues 1-48)

The fragments GroEL(191-345) or GroEL(191-376) are expressed in E. coli with a 17-residue N-terminal tail containing 6 histidine residues so that they could be purified using Ni-NTA resin—the Ni²⁺ ion which is chelated by the agarose binds to the histidine tag. Initially, the same resin is used to immobilise purified GroEL(191-345) or GroEL(191-376) by their histidine tags for preparative purposes. Subsequently, the purified fragments are also linked covalently to agarose via CNBr activation .(Axen R et al (1967) Nature 1302-1304). The immobilised Gro.EL fragments are found to facilitate the refolding of cyclophilin A with high efficiency.

The apical domain of GroEL (GroEL(191-376)) and the. “core” of the apical domain, GroEL(191-345), are cloned and expressed in E. coli as fusion proteins containing a 17-residue N-terminal histidine-tail. The fragments are immobilised by two methods.

A). Immobilised apical domain attached to Ni-NTA resin.

The Ni-NTA resin (from QIAGEN) is a chelating adsorbant composed of a high surface concentration of nitrilo-tri-acetic acid (NTA) ligand attached to Sepharose CL-6B. The NTA occupies four of six ligand binding sites in the coordination sphere of the Ni²⁺ ion, leaving two sites free to interact with the six histidines in the N-terminal tail. Proteins containing one or more 6× His affinity tags, located at either the amino or carboxyl terminus of the protein, bind to the Ni-NTA resin with an affinity (K_{d}=10-13 M, pH 7.8). The stability of the 6×His/Ni-NTA interaction is unaffected by strong denaturants such as 6M guanidine hydrochloride or 8M urea, or the presence of low levels of B-mercaptoethanol (1-10 mM). 3.5 mnL of Ni-NTA resin are equilibrated with 0.1M potassium phosphate at pH 7.8, containing 5 mM β-mercaptoethanol. The GroEL domain is added to saturation of the affinity gel (21 mg of protein per 3.5 mL of gel) and incubated at room temperature for 30 min with gentle mixing. The gel is packed in a column suitable for FPLC (5×100 mm, from Pharmacia) and thoroughly washed with the initial buffer.

B). Immobilised apical domain attached to CNBr-activated Sepharose 4B

To minimise the steric effects and preserve the structure of the binding site in the apical domain, the binding capacity of the gel is reduced by controlled hydrolysis of the activated gel before coupling. 300 mg of freeze-dried powder are suspended in 50 mM of NaHCO₃ pH 8.3, washed with the same buffer and re-swollen on a sintered glass filter (G3), then suspended in the buffer and mixed in an end-over-end shaker for 4 h at room temperature. The apical domain, dissolved in the coupling buffer (O.M NaHCO₃, pH 8.3 and 0.5 M NaCl), is added to the gel suspension (10 mg protein/mL gel) and mixed in an end-over-end shaker for 6 h at room temperature. It is then washed with the coupling buffer. The remaining active groups are blocked by addinz 2.5 M ethanolamine pH 8 and shaking for 4 h at room temperature. Uncoupled apical domain is removed by washing with five cycles of alternately high and low pH buffer solution (Tris-HCl 0.1M pH 7.8 containing 0.5M NaCl followed by acetate buffer (0.1 M, pH 4 plus 0.5M NaCi). The gel is finally washed with 0.1 M potassium phosphate at pH 7.8, containing 5 mM 2-mercaptoethanol (refolding buffer).

C). Immobilised apical domain attached to NHS-Sepharose

Ligand coupling. NHS-activated sepharose ( 5 ml ) is unpacked from a HiTrap NHS-activated column and washed extensively (50 ml) with 1 mM HCl, ice cold, using a Buchener funnel. The binding solution (mini-chaperone) can then be added to the slurry in the desired concentration (10 mg/ml).

Binding solution: minichaperone (10 mg/ml), in NaHCO₃ pH 9, 0.5M NaCl, is gently mixied and then a saturated solution of sodium sulfate is added, until it just starts to become cloudy oust before the concentration reches the precipitation point). When reacting the support. it is necessary to gently agitate the reaction tubes by tumbling for 20 hrs at room temperature. Once immobilization and any quenching reactions are complete, the support is washed extensively first with coupling buffer, then with a high salt (1M NaCl) buffer to eliminate licand that may be bound to the support through protein/protein interactions.

Excess groups are blocked with 1M ethanolamine for at least 4 hr or overnight, at RT, then the Sepharose is washed with bicarbonate buffer (0.1M,pH 9.2) and acetate buffer (0.1M pH 4.0), before a final wash in refolding buffer.

The immobilised fragments are used in a chromatography column (FIG. 6). Denatured protein in urea is added to the column and developed with a refolding buffer. The protein eluted as from a conventional binding column, its passage is retarded and the peak could be characterised by a retention time. Some mutants of indoleglycerol phosphate synthase (IGPS) are expressed E. coli and isolated as inclusion bodies. These reprecipitate on attempts to renature them after dissolving at high concentrations in urea, and attempts at low protein concentrations yielded soluble material of non-native conformation. The mutant IGPS (49-252) (1×22 kDa), which lacks the first 48 amino acid residues, is obtained on chromatography on the GroEL(191-345) column in a 92% yield of material that have 100% binding activity (FIG. 6). The term “refolding chromatography” can be used to describe the phenomenon of refolding by passage through the column.

In more detail, a column of ht-GroEL 191-345 immobilised on Ni-NTA agarose (3.0 mrL) is loaded with 2 nmol of IGPS (49-252) dissolved in 20 μL of 8 M urea, and the column is developed with refolding buffer (0.1 M potassium phosphate at pH 7.8, containing 5 mM 2-mercaptoethanol) using a Waters 625 LC HPLC system. In FIG. 6, the two peaks are seen which are described by: Kₐᵥ=(V₃−Vₒ)/(Vₜ−Vₒ), where Vₜ is the total volume of gel in the column, Vₒ, the void volume, and Ve the volume at the maximum of the peak. A value of Kₐᵥ indicates that the protein interacts with the support. Peak 1 have Kₐᵥ=1.0 (9.6% of the total area) and peak 2 Kₐᵥ=1.7 (90.4%). The protein in peak 2 is recovered in 1.2 mL, and contained 1.85 nmol of truncated IGPS (92.5% yield). It displays a circular dichroism spectrum characteristic of a native alb protein and bound ³H-rCdRP (tritium-labelled reduced 1 [(2-carboxyphenyl)amino]-1-deoxyribulose 5-phosphate), a specific inhibitor of IGPS, with a stoichiometry of 1.0. Initially denatured cyclophilin chromatographs with two peaks, an inactive at Kₐᵥ=0.38 and an active at Kₐᵥ1.43

### EXAMPLE 9

Batchwise renaturation of cyclophilin A on Ni-NTA agarose gels

This experiment uses batchwise mixing of materials. A solution of denatured cyclophilin in 8 M urea is diluted 100 fold with Ni-NTA-imnnobilised GroEL(191-345) in a refolding buffer (see Table II below). After gentle mixing for 30 min, the cyclophilin is recovered in 84% yield of protein. Control experiments (Table II) in which the protein is denatured in 8 M urea and added to the refolding buffer alone, or mixed with agarose that have not been linked to GroEL fragments, gave cyclophilin that have only 20% of the activity of the starting material before denaruration. The cyclophilin used for the denaturation experiments have 88% of the activity of the purest samples previously obtained by the inventors. The specific activity of the material obtained from the GroEL(191-345)-agarose resin (covalently linked) is 126% of that of the previous purest samples. Further, the total recovery of activity is 25% more than that initially present. Thus, the irnmobilised GroEL fragment have “reconditioned” the cyclophilin A by converting inactive material into active.

In more detail, suspension of 200 μL of gel (wet, sedimented volume) is mixed with refolding buffer (100 mnM phosphate buffer, pH 7.8 plus 5 mM 2-mercaptoethanol) to give a volume of 990 μL. 10 μL of cyclophilin (from a 100 μM stock solution in refolding buffer+8M urea,=1 nmol of cyclophilin A) are added and the suspension is mixed in an up-down mixer for 30 min at room temperature.

The gel suspension is centrifuged to separate the supernatant (˜800 μL). The gel pellet is washed in miniprep columns and the eluate added to the supernatant to give about 900 μL. The protein concentration is determined from the A₂₈₀ nm. Cyclophilin activity is measured in the supernatant as described in (Makino Y et al (1993) FEBS Lett 336: 363-367). The sample prior to denaturation have 88% of the specific activity of the highest activity of native cyclophilin previously obtained by the inventors. The control is agarose alone, without Ni. The results are shown in table II below.

TABLE II

| | | Cyclophilin | Cyclophilin specific |
|---|---|---|---|
| | Protein | activity | activity relative |
| | yield | yield | to “pure” native |
| Gel type | (%) | (%) | (%) |
| GroEL 191-345 agarose | 84 | 98 | 103 |
| GroEL 191-376 agarose | 81 | 105 | 115 |
| GroEL 191-345 agarose | 87 | 125 | 126 |
| Agarose (control)⁵ | 72 | 17 | 20 |
| Native cyclophilin (control) | | | 88 |

The procedure is applied to other proteins used in the laboratory. Glucosamine 6-phosphate deaminase (6×30 kDa) (Oliva G et al (1995) Structure a: 1323-1332) normally regains only 10% or less activity after renaturation from urea denaturation. A 100% yield is obtained on batchwise treatment with GroEL(191-345)-agarose. Further, a sample that have lost all activity on storage in solution in 50% glycerol/water at −20° C. for 5 years also regained 100% activity with this treatment after dissolving in urea.

### EXAMPLE 10

Residues of GroEL implicated in binding peptides

The X-ray crystal structure of GroEL 191-376 with the 17 residue N-tenninal tail shows that seven residues of the tail of one molecule bind in the active sire of the other. Residues 230-271 are in the binding site. All residues are shown in Table III in which large, bold and underlined residues are those detected by X-ray crystal structure of ht GroEL 191-376 as being involved in protein binding.

The X-ray crystal structure shows that 193-336 should be reasonably stable. The 193-337 fragment is cloned and expressed and found to be stable. Therefore, residues 191 and 192 may be omitted.

TABLE III

| | |
|---|---|
| 190 | VEGMQFDRGY LSPYFINKPE TGAVELESPF ILLADKKISN |
| 230 | IREMLPVLEA VAKAGKIPLLI IAEDVEGEAL ATLVVNTMRG |
| 270 | IVKVAAVKAP GPGDRRKAML QDIATLTGGT VISEEIGMEL |
| 310 | EKATLEDLGQ AKRVVINKDT TTIIDGVGEE AAIQGRVAQI |

### EXAMPLE 11

Construction and testing of minimal minichaperone fragments

Various N and C terminally truncated fragments of the apical domain of GroEL, includoing fragment 193-335, are cloned by PCR into BamRI and EcoRI sites of the pRSET A vector (Invitrogen) encoding an N-terminal histidine tail (17 amino acids; “sht”) which comprises an engineered thrombin cleavage site (Zahn. et al., (1996) PNAS (USA) 93:15024-15029). Plasmid construction is verified by PCR cycle sequencing using fluorescent dideoxy chain terminators according to the manufacturer's instructions (Applied Biosystems). Sequencing reactions are analysed on an Applied Biosystems 373A Automated DNA Sequencer.

The overexpression and purification of the minichaperone in E. coli BLR(DE3) cells is carried out essentially as previously described (Zahn et al., Szpra). sht-GroEL(193-335) is over-expressed in BLR(DE3) cells to give ˜100 mg purified protein per litre of culture. The GroEL fragments are analysed by quantitative amino acid analysis. N-ter-minal sequencing, and mass spectrometry. Protein concentration is determined by absorbance at 276 nm using an extinction coefficient calculated from Gill & von Hippel, (1989) Anal. Biochem. 182:319-326. In order to assess the degree of association between the fragments, dynamic light scattering experiments are performed at 25° C. with a DynaPro-801TC Molecular Sizing instrument (Protein Solutions, Inc., Charlottesville, VA, USA). The protein concentrations used are in the 1M range in 50 mM Tris-HCl, 150 mM NaCl, pH 8.2 buffer. sht-GroEL(193-335) purified by gel-filtration is monomeric at μM concentrations, as determined by light scattering experiments.

Refolding assays of rhodanese and cyclophilin A are performed as above. sht-GroEL 193-335 is as active in vitro as sht-GroEL(191-345) and sht-GroEL(191-376) in chaperoning the folding of rhodanese and cyclophilin A.

### EXAMPLE 12

AlignTment of consensus binding sequences of cpn60 family members

FIG. 9 shows sequences in OWL database release 28.1 containing clear homology to apical domain of GroEL (residues 191-375) in PDB structure pdblgrl.ent. OWL is a non redundant database merging SWISS-PROT, PIR (1-3), GenBank (translation) and NRL-3D. Consensus sequence=residues 230-271 inclusive) containing peptide-binding site (as identified by crystal structure analysis and polypeptide binding studies). X=residue in peptide-binding site in the X-ray crystal structure of mini chaperone. The GroEL E. coli chaperone sequence is shown in italics.

1. A chaperone polypeptide having an amino acid sequence selected from at least amino acid residues 230-271 but no more than residues 150455 or 151456 of a GroEL sequence substantially as shown in FIG. 7, or a corresponding sequence of a substantially homologous chaperone polypeptide, or a modified mutated or variant sequence thereof having chaperone activity.

2. Monomeric polypeptide having chaperone activity and incapable of multimerisation in solution.

3. A chaperone polypeptide which when in solution, remains monomeric and has the ability to refold, reactivate or recondition proteins said polypeptide including the protein binding active site motif:
1 X X X 2 X X 3 4 X X 5 X X X X X X X X X X X X X X X 6 X X 7 8 X 9 10 11 X X 12 X 13 14
wherein 1 is selected from amino acid residues:
I, M, L, V, S, F or A;
wherein 2 is selected from: L, I, P, V or A;
wherein 3 is selected from: L, E, V, H or 1;
wherein 4 is selected from: E, A, R, L, Q, or N;
wherein 5 is selected from: A, V, I, M, L, N, S, R, T, Q or K;
wherein 6 is selected from: E, D or G;
wherein 7 is selected from: A, P, S, T, G or L;
wherein 8 is selected from: T, A; N, S or V;
wherein 9 is selected from: V, L, I or A;
wherein 10 is selected from: V, L, I, F or H;
wherein 11 is selected from: N, S or L;
wherein 12 is selected from: R, K, N, Q, L or S;
wherein 13 is selected from: I, T, S, G, V, A, Q, N, K, F or P;
wherein 14 is selected from: V, I, L, F, D or T; and
wherein the Xs represent a peptide bond or bonds or at least one amino acid residue,
or a functional variant thereof in which one or more of the numbered amino acid residues 1 to 14 has undergone a conservative substitution.

4. A chaperone polypeptide which, when in solution, remains monomeric and has the ability to refold reactivate or recondition proteins, said polypeptide including at least one protein binding active site motif moiety selected from:
(a) 1 X X X 2 X X 3 4 X and
(b) X 5 X X X X X X X X X X X X X X X 6 X X 7 8 X 9 10 11 X X 12 X 13 14
wherein 1 is selected from amino acid residues:
I, M, L, V, S, F or A;
wherein 2 is selected from: L, I, P, V or A;
wherein 3 is selected from: L, E, V, H or 1;
wherein 4 is selected from: E, A, R, L, Q, or N;
wherein 5 is selected from: A, V, I, M, L, N, S, R, T, Q or K;
wherein 6 is selected from: E, D or G;
wherein 7 is selected from: A, P, S, T, G or L;
wherein 8 is selected from: T, A, N, S or V;
wherein 9 is selected from: V, L, I or A;
wherein 10 is selected from: V, L, I, F or H;
wherein 11 is selected from: N, S or L;
wherein 12 is selected from: R. K, N, Q, L or S;
wherein 13 is selected from: I, T, S, G, V, A, Q, N, K, F or P;
wherein 14 is selected from: V, I, L, F, D or T; and
wherein X is at least one amino acid residue,
or a functional variant thereof in which one or more of the numbered amino acid residues 1 to 14 has undergone a conservative substitution.

5. A chaperone polypeptide which when in solution, remains monomeric and has the ability to refold, reactivate or recondition proteins, said polypeptide including the protein binding active site motif:
I X X X L X X L E X X A X X X X X X X X X X X X X X X E X X A T X V V N X X R X I V
wherein X is at least one amino acid residue, or a functional variant thereof in which one or more of the specified amino acid residues has undergone a conservative substitution.

6. A chaperone polypeptide which when in solution, remains monomeric and has the ability to refold, reactivate or recondition proteins, said polypeptide including at least one protein binding active site motif moiety selected from:
(a) I X X X L X X L E X
(b) X A X X X X X X X X X X X X X X X E X X A T X V V N X X R X I V
wherein X is at least one amino acid residue, or a functional variant thereof in which one or more of the specified amino acid residues has undergone a conservative substitution.

7. A chaperone polypeptide as claimed in any one of claims 3 to 6 in which the active site motif or an active site motif moiety includes the conserved sequence:
P L L (V) I (V) I A (S) E D V (I) E G E A L
in which amino acid symbols in parenthesis are alternatives to the immediately preceding symbol reading left to right.

8. Monomeric polypeptide having chaperone activity and incapable of multimerisation characterised in that in the absence of ATP the polypeptide has a protein refolding activity of more than 50%, preferably 60%, even more preferably 75%, said refolding activity being determined by contacting the polypeptide with an inactivated protein of known specific activity prior to inactivation, and then determining the specific activity of the said protein after contact with the polypeptide, the % refolding activity being:
$$\frac{specific activity of protein after contact with polypeptide}{specific activity of protein prior to inactivation} \times \frac{100}{1}$$

9. A polypeptide as claimed in any preceding claim, wherein the chaperone activity is determined by the refolding of cyclophilin A.

10. A polypeptide as claimed in claim 9 wherein 8M urea denatured cyclophilin A (100 μM) is diluted into 100 mM potassium phosphate buffer pH7.0, 10 mM DTT to a final concentration of 1 μM and then contacted with at least 1 μM of said polypeptide at 25° C. for at least 5 min, the resultant cyclophilin A activity being assayed by the method of Fischer G et al (1984) Biomed Biochim Acta 43: 1101-1111.

11. A polypeptide as claimed in any preceding claim being an hsp60 polypeptide, preferably a GroEL polypeptide.

12. A polypeptide as claimed in any preceding claim which comprises at least an amino acid sequence selected from GroEL residues:
(a) 191-329, 191-330, 191-331, 191-332, 191-333, 191-334, 191-335, 191-336, 191-337, 191-338, 191-339, 191-340. 191-341, 191-342, 191-343, 191-344, 191-345, 191-346, 191-347, 191-348, 191-349, 191-350, 191-351, 191-352, 191-353, 191-354, 191-355, 191-356, 191-357, 191-358, 191-359, 191-360, 191-361, 191-362, 191-363, 191-364, 191-365, 191-366, 191-367, 191-368, 191-369, 191-370, 191-371, 191-372, 191-373, 191-374, 191-375 or 191-376, or
(b) 192-329, 192-330, 192-331, 192-332, 192-333, 192-334, 192-335, 192-336, 192-X37, 192-338, 192-339, 192-340, 192-341, 192-342, 192-343, 192-344, 192-345, 192-346, 192-347, 192-348, 192-349, 192-350, 192-351, 192-352, 192-353, 192-354, 192-355, 192-356, 192-357, 192-358, 192-359, 192-360, 192-361, 192-362, 192-363, 192-364, 192-365, 192-366, 192-367, 192-368, 192-369, 192-370, 192-371, 192-372, 192-373, 192-374, 192-375 or 192-376, or
(c) 193-329, 193-330, 193-331, 193-332, 193-333, 193-334, 193-335, 193-336, 193-337, 193-338, 193-339, 193-340, 193-341, 193-342, 193-343, 193-344, 193-345, 193-346, 193-347, 193-348, 193-349, 193-350, 193-351, 193-352, 193-353, 193-354, 193-355, 193-356, 193-357, 193-358, 193-359, 193-360, 193-361, 193-362, 193-363, 193-364, 193-365, 193-366, 193-367, 193-368, 193-369, 193-370, 193-371, 193-372, 193-373, 193-374, 193-375 or 193-376, or
(d) 230-271, 229-271, 229-272, 228-272, 228-273, . . . et seq . . . 194-328, 194-329, or
the equivalent residues of substantially homologous chaperoning, or a modified, mutated or variant sequence thereof.

13. A polypeptide as claimed in claim 8, wherein the selected amino acid sequence is selected from the group consisting of 230-271, 191-345. 191-376, 193-335 and 193-337 of GroEL, the equivalent residues of substantially homologous chaperoning, and a modified, mutated or variant sequence thereof.

14. A polypeptide as claimed in any preceding claim further comprising a polyamino acid sequence, preferably an N-terminal polyamino acid sequence.

15. A polypeptide as claim in claim 14, wherein the polyamino acid sequence is a polyhistidine sequence.

16. A polypeptide as claimed in claim 14 or claim 15, wherein the polyamino acid sequence includes a cleavage site cleavable by a cleavage agent, preferably said cleavage agent is thrombin.

17. A polypeptide as claimed in any one of claims 14 to 16 wherein the further polyamino acid sequence comprises a number of amino acid residues in the range 2 to 500, preferably 5 to 100, more preferably 17 to 39.

18. A polypeptide as claimed in any preceding claim and is immobilised form, optionally immobilised to a chromatographic matrix, preferably an agarose resin.

19. A polypeptide as claimed in claim 18, wherein the agarose resin is a nickel-nitrilo-tri-acetic acid (NTA)-ligated agarose resin.

20. A polypeptide as claimed in any preceding claim fused to a heterologous protein or polypeptide.

21. A recombinant polypeptide as claimed in any preceding claim.

22. An isolated nucleic acid molecule comprising a nucleotide sequence encoding a polypeptide as defined in any one of claims 1 to 21 or a nucleotide sequence hybridisable thereto and optionally encoding a polypeptide having chaperone activity.

23. A recombinant nucleic acid molecule for use in cloning and/or expressing a nucleic acid sequence, said recombinant nucleic acid molecule comprising:
(a) a nucleotide sequence encoding amino acid residues 191-376 of GroEL, or
(b) a nucleotide sequence encoding amino acid residues 191-345 of GroEL, or
(c) a nucleotide sequence encoding amino acid residues 193-337of GroEL, or
(d) a nucleotide sequence encoding amino acid residues 193-335of GroEL, or
(e) a nucleotide sequence encoding amino acid residues of GroEL selected from amino acid residues:
(i) 191-329, 191-330, 191-331, 191-332, 191-333, 191-334, 191-335, 191-336, 191-337, 191-338, 191-339, 191-340, 191-341, 191-342, 191-343, 191-344, 191-345, 191-346, 191-347, 191-348, 191-349, 191-350, 191-351, 191-352, 191-353, 191-354, 191-355, 191-356, 191-357, 191-358, 191-359, 191-360, 191-361, 191-362, 191-363, 191-364, 191-365, 191-366, 191-367, 191-368, 191-369, 191-370, 191-371, 191-372, 191-373, 191-374, 191-375 or 191-376, or
(ii) 192-329, 192-330, 192-331, 192-332, 192-333, 192-334, 192-335, 192-336, 192-337, 192-338, 192-339, 192-340, 192-341, 192-342, 192-343, 192-344, 192-345, 192-346, 192-347, 192-348, 192-349, 192-350, 192-351, 192-352, 192-353, 192-354, 192-355, 192-356, 192-357, 192-358, 192-359, 192-360, 192-361, 192-362, 192-363, 192-364, 192-365, 192-366, 192-367, 192-368, 192-369, 192-370, 192-371, 192-372, 192-373, 192-374, 192-375 or 192-376, or
(iii) 193-329, 193-330, 193-331, 193-332, 193-333, 193-334, 193-335, 193-336, 193-337, 193-338, 193-339, 193-340, 193-341, 193-342, 193-343, 193-344, 193-345, 193-346, 193-347, 193-348, 193-349, 193-350, 193-351, 193-352, 193-353, 193-354, 193-355, 193-356, 193-357, 193-358, 193-359, 193-360, 193-361, 193-362, 193-363, 193-364, 193-365, 193-366, 193-367, 193-368, 193-369, 193-370, 193-371, 193-372, 193-373, 193-374, 193-375 or 193-376, or
(f) 230-271, 229-271, 229-272, 228-272, 228-273, . . . et seq . . . 194-328, 194-329, or
(g) a nucleotide sequence hybridisable to any of (a), (b), (c), (d), (e) or (f) above and encoding a monomeric polypeptide having chaperone activity, or
(h) degenerate nucleotide sequences corresponding to (a), (b), (c), (d), (e), (f) or (g) above.

24. A vector comprising a nucleic acid as claimed in claim 22 or claim 23.

25. A host cell transformed with a nucleic acid as defined in claim 22 or claim 23 or vector as defined in claim 24.

26. A method of making a polypeptide as defined in any one of claims 1 to 21 comprising transforming a host cell with a nucleic acid encoding said polypeptide, culturing the transformed cell and expressing said polypeptide.

27. A method of making a polypeptide as claimed in claim 26 wherein the nucleic acid is as defined in claim 22 or claim 23.

28. A method as claimed in claim 26 or claim 27, wherein the expressed polypeptide product is subject to cleavage.

29. A pharmaceutical formulation comprising a polypeptide of any one of claims 1 to 21, optionally together with a diluent, carrier or excipient.

30. A polypeptide as defined in any one of claims 1 to 21 for use in the treatment of disease.

31. The use of a polypeptide as defined in any one claims 1 to 21 in the manufacture of a medicament for the treatment of disease associated with protein/polypeptide structure.

32. A nucleic acid molecule as defined in claims 22 or claim 23 for use in the treatment of disease.

33. The use of nucleic acid molecule as defined in claim 22 or claim 23 in the manufacture of a medicament for the treatment of disease associated with protein polypeptide structure.

34. A method of reconditioning a molecule preferably a protein comprising contacting said protein with a polypeptide of any one of claims 1 to 21.

35. A method as claimed in claim 34, wherein the protein is subjected to inactivation or denaturation prior to contacting with said polypeptide.

36. A method as claimed in claim 34 or claim 35, wherein the polypeptide is immobilised to a solid phase.

37. A method as claimed in claim 33 or claim 34, wherein the polypeptide is immobilised to a solid phase, preferably a chromatographic matrix, and the contacting of protein and polypeptide is carried out by applying the protein to the top of a bed of the matrix packed in a column and then eluting the polypeptide through the column.

38. Use of a polypeptide as claimed in any one of claims 1 to 21 for altering the structure of a molecule.

39. The use of claim 38, wherein the molecule is a protein or polypeptide and the alteration in structure is by folding unfolding or refolding.

40. The use of claim 38 or claim 39, wherein the stoichiometry between the polypeptide and the molecule being altered is about 1:1.

41. Use of a polypeptide as defined in any one of claims 1 to 21 in the purification or increase in yield, specific activity or quality of biological molecules, preferably said polypeptide being attached to a support.

42. A kit for reconditioning or refolding a molecule preferably a protein, comprising a polypeptide of any one of claims 1 to 21 immobilised to a solid phase and a container for holding said solid phase polypeptide.

43. Use of a polypeptide as defined in any one of claims 1 to 21 in the production of a protein or polypeptide by recombinant means, wherein the said polypeptide is co-expressed with the protein or polypeptide thereby to improve the yield or quality of the protein or polypeptide.

44. An antibody reactive against a polypeptide as defined in any one of claims 1 to 21.

45. An antibody as claimed in claim 44 for use in the treatment of disease.

46. The use of an antibody as claimed in claim 44 in the manufacture of a medicament for the treatment of disease associated with protein/polypeptide structure.

47. A method of treating disease in which an effective amount of a polypeptide of any one of claims 1 to 21 is administered.

48. A method of treating disease which comprises administering an effective amount of an inhibitor of the chaperone activity of a polypeptide of any one of claims 1 to 21.

49. A method as claimed in claim 48 wherein said inhibitor is an antibody.

50. A method of treating disease by gene therapy which utilises a construct encoding a polypeptide of any one of claims 1 to 20 or an antagonist thereof.
